(19) United States
(12) Patent Application Publication
(10) Pub. No.: US 2025/0099263 A1
(43) Pub. Date: Mar. 27, 2025

(54) ROTATABLE AND SURGICAL APPROACH-SPECIFIC INTERVERTEBRAL IMPLANTS FOR FUSION TECHNIQUES

(71) Applicant: Carlsmed, Inc., Carlsbad, CA (US)

(72) Inventors: Kyle Seaman, Vista, CA (US); Jeremy Winston, San Diego, CA (US); Austin Wulf, Carlsbad, CA (US)

(21) Appl. No.: 18/892,151

(22) Filed: Sep. 20, 2024

Related U.S. Application Data
(60) Provisional application No. 63/539,797, filed on Sep. 21, 2023.

(51) Int. Cl. A61F 2/44; A61F 2/30; A61F 2/46

(52) U.S. Cl. CPC A61F 2/4465; A61F 2/4611; A61F 2002/30133; A61F 2002/4629

(57) ABSTRACT

A spinal fusion system includes an intervertebral implant. The intervertebral implant has a fusion body configured to contact endplates of adjacent vertebrae and an instrument coupler. The instrument coupler is detachably couplable to an inserter instrument. When the inserter instrument extends through the instrument coupler and contacts the fusion body, the intervertebral implant is rigidly coupled to the inserter instrument. When the inserter instrument is spaced apart from the fusion body, the fusion body is rotatable relative to the inserter instrument while the instrument coupler holds the inserter instrument.

### CROSS-REFERENCE TO RELATED APPLICATION(S)

This application claims priority to U.S. Provisional Application No. 63/539,797, filed Sep. 21, 2023 which is hereby incorporated by reference in its entirety.

### TECHNICAL FIELD

The present invention relates generally to articulating implants, approach-specific intervertebral implants, and surgical techniques. More specifically, the invention relates to rotatable intervertebral implants for spinal procedures, including fusion procedures, such as transforaminal lumbar interbody fusion (TLIF) procedures, approach-specific implants, and delivery instruments.

### BACKGROUND OF TECHNOLOGY

Orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis. Unfortunately, it may be difficult to implant spinal implants at targeted locations using a desired delivery path.

### SUMMARY

At least some embodiments are directed to rotatable intervertebral implants for spinal procedures. The implants can be rigidly attached to an inserter instrument, and a body of the implant can rotate relative to the inserter instrument to allow complex navigation within the patient's body. In TLIF procedures, implants (e.g., C-shaped implants, crescent shaped implants, etc.) can be inserted into a disc space at an oblique angle. Once the implant crosses a midline of the disc space, a cage or body of the implant can be rotated in the anterior direction, such that the cage main body (e.g., fusion body) is positioned along targeted bone, such as cortical bone of the vertebrae. A user can reengage the inserter instrument with the implant for repositioning and/or removal of the implant. The implant can be C-shaped TLIF implant designed to be implanted transversely across the patient's midsagittal plane.

The implant can include the instrument coupler with a slidable thread receiver positioned inwardly of a periphery of the implant such that movable components are not exposed to the patient's disc space. This helps limit or prevent healthy tissue becoming caught by moving implements of the implant. The cage can include patient-matched contoured endplates for contacting adjacent vertebral bodies. The instrument coupler can be positioned directly between the contoured endplates. The movable component(s) of the implant can be positioned inwardly of the periphery of the implant body.

In some embodiments, a spinal fusion system includes an intervertebral implant having a fusion body and an instrument coupler. The fusion body is configured to contact endplates of adjacent vertebrae. The instrument coupler is detachably couplable to an inserter instrument, such that when the inserter instrument extends through the instrument coupler and contacts the fusion body, the fusion body is rigidly coupled to the inserter instrument. When the inserter instrument is spaced apart from the fusion body, the fusion body is movable relative to the inserter instrument, which remains held by the instrument coupler. The instrument coupler of the implant can be stationary (e.g., rotationally and/or translationally stationary) with respect to the inserter instrument as the fusion body of the implant rotates or pivots relative to the inserter instrument. For example, a distal end of the fusion body can rotate away from the longitudinal axis of the inserter instrument radially outward relative to the inserter instrument and/or an insertion or delivery path. The fusion body can be configured to receive and hold material. Additionally or alternatively, the instrument coupler can be configured to detachably couple the implant to the inserter instrument (or delivery instrument) such that the implant is angled with respect to an axis (e.g., the longitudinal axis) or plane (e.g., midplane, sagittal plane, etc.) of the insertion instrument. For example, the instrument coupler can position the implant at a target location along a delivery path for a planned surgical approach. The relationship between the implant and inserter instrument can be selected based on, for example, delivery path to a target implantation site, an insertion path between anatomical elements, a surgical technique, visualization techniques, etc.

In some embodiments, a system can be used to design and manufacture implants with printed bodies, sliding parts, and/or other components (e.g., instruments). In some embodiments, implants are printed in-place and do not require any post-print assembly operations. For rotating implants, rotational limits of implants may be customized for each patient or may be set as a fixed parameter of the design. The rotational limits can be incorporated into a surgical plan reviewable by a physician. In some embodiments, sliding thread parts of intervertebral cages can be nested and not exposed to the patient's disc space, and the cages can incorporate patient-matched endplate contours.

### BRIEF DESCRIPTION OF THE DRAWINGS

Non-limiting and non-exhausting embodiments are discussed with reference to the following drawings. The same reference numerals refer to like parts or acts throughout the various views, unless specified otherwise.

FIG. 1 is a side view of two implants located along a patient's spinal column and configured in accordance with embodiments of the present technology.

FIG. 2 is a superior-inferior view of an implant located along a vertebral body of FIG. 1 in accordance with embodiments of the present technology.

FIG. 3 is an isometric view of a rotatable implant in a delivery configuration in accordance with an embodiment of the present technology.

FIG. 4 is an isometric view of the implant of FIG. 3 in a deployed configuration in accordance with an embodiment of the present technology.

FIG. 5 is an isometric view of a rotatable implant in accordance with an embodiment of the present technology.

FIG. 6 is a side view of the implant of FIG. 5.

FIG. 7 is a front view of the implant of FIG. 5.

FIG. 8A is a cross-sectional view of the implant taken along line 8A-8A of FIG. 7 in the delivery configuration.

FIG. 8B is a cross-sectional view of the implant of FIG. 8A in the deployed configuration.

FIG. 9 is an exploded isometric view of a surgical system in accordance with an embodiment of the present technology.

FIG. 10 is an isometric view of an instrument coupler in accordance with an embodiment of the present technology.

FIG. 11 is a top view of the instrument coupler of FIG. 10.

FIG. 12 is a side view of the instrument coupler of FIG. 10.

FIG. 13 is a front view of the instrument coupler of FIG. 10.

FIG. 14 is a cross-sectional view of the instrument coupler taken along line 14-14 of FIG. 13.

FIGS. 15-18 show a method for implanting a rotatable implant in accordance with embodiments of the present technology.

FIG. 19A is a schematic top plan view showing surgical approaches for performing spinal procedures.

FIG. 19B is an isometric view of the spine of FIG. 19A.

FIGS. 20 and 21 are isometric views of an implant with a pivoting instrument coupler in accordance with an embodiment of the present technology.

FIGS. 22 and 23 are isometric and front views, respectively, of a ratcheting implant and an inserter instrument in accordance with an embodiment of the present technology.

FIG. 24 is a network connection diagram illustrating a system for providing patient-specific medical care and configured in accordance with select embodiments of the present technology.

FIG. 25 shows an implant and a delivery/insertion instrument for an anterior lateral lumbar interbody fusion (ALIF) approach in accordance with at least some embodiments of the technology.

FIG. 26 shows the implant of FIG. 25 at a target implant position in accordance with at least some embodiments of the technology.

FIG. 27 is an isometric view of an implant in accordance with at least some embodiments of the technology.

FIG. 28 is a front view of the implant of FIG. 27.

FIG. 29 is a cross-sectional view of the implant of FIG. 27 taken along line 29-29 of FIG. 28.

FIGS. 30 and 31 are cross-sectional views showing an insertion instrument and an implant.

FIG. 32 is an isometric view of an implant in accordance with at least some embodiments of the present technology.

FIG. 33 is a top view of the implant of FIG. 32.

FIG. 34 is a front view of the implant of FIG. 32.

FIG. 35 is a cross-sectional view of the implant taken along line 35-35 of FIG. 34.

FIGS. 36 and 37 are cross-sectional views showing an insertion instrument and an implant.

FIG. 38 shows anatomy and an implant designed for a high iliac crest lateral lumbar interbody fusion (LLIF) procedure in accordance with at least some embodiments of the technology.

FIG. 39 is an isometric view of the implant of FIG. 38 in accordance with at least some embodiments of the present technology.

FIG. 40 is a bottom view of the implant of FIG. 38.

FIG. 41 is a front view of the implant of FIG. 38.

FIG. 42 is a cross-sectional view of the implant of FIG. 38 taken along line 42-42 of FIG. 41.

FIGS. 43 and 44 are cross-sectional views showing an insertion instrument coupling to the implant of FIG. 39.

FIG. 45 is a front view of anatomy and an implant designed for a low rib LLIF procedure in accordance with at least some embodiments of the technology.

FIG. 46 is an isometric view of an implant in accordance with at least some embodiments of the present technology.

FIGS. 47 and 48 are cross-sectional views showing an insertion instrument coupling to the implant.

FIG. 49 is a top view of anatomy and an implant designed for an oblique lumbar interbody fusion (OLIF) procedure in accordance with at least some embodiments of the technology.

FIG. 50 is an isometric view of an implant in accordance with at least some embodiments of the present technology.

FIG. 51 is a side view of the implant of FIG. 50.

FIG. 52 is a cross-sectional view of the implant taken along line 52-52 of FIG. 51.

FIGS. 53 and 54 are cross-sectional views showing an insertion instrument coupling to the implant.

FIG. 55 is a top view of anatomy and an implant designed for an ALIF/OLIF procedure in accordance with at least some embodiments of the technology.

FIG. 56 shows an implant of FIG. 55 in accordance with at least some embodiments of the technology.

FIGS. 57 and 58 are opposite side views of the implant of FIG. 56.

FIG. 59 is a front view of the implant of FIG. 56.

FIG. 60 is a cross-sectional view of the implant taken along line 60-60 of FIG. 59.

FIGS. 61-64 are cross-sectional views showing the insertion instrument coupling to the different coupling features of the implant of FIG. 55 in accordance with at least some embodiments of the technology.

FIGS. 65-67 are flow diagrams illustrating example processes for designing a surgical approach-specific implant in accordance with at least some embodiments of the technology.

### DETAILED DESCRIPTION

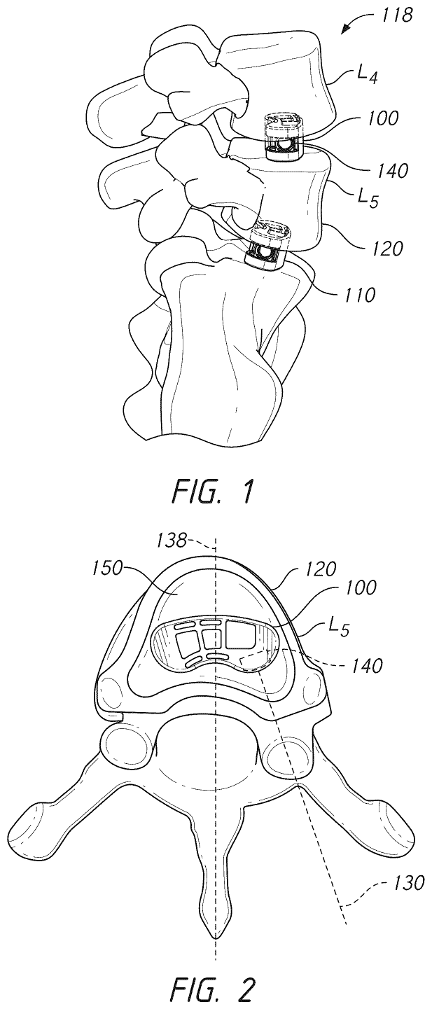

FIG. 1 is a side view of implants 100, 110 located along a patient's spinal column 118 and configured in accordance with embodiments of the present technology. FIG. 2 is a superior-inferior view of the implant 100 located in an intervertebral disc space and resting on a vertebral body 120 (illustrated as an L₅ vertebral body). The implant 100 can articulate relative to an inserter instrument until the implant 100 is positioned at a target position. In some procedures, the implant 100 and inserter instrument can be arranged in-line or in a linear configuration for delivery into the patient. After the implant 100 is positioned at the target vertebral level, the inserter instrument can be operated to unlock the implant 100 so that a body of the implant 100 is capable of moving (e.g., articulating, rotating, translating, etc.) relative to the inserter instrument. Once the implant 100 is at the target location, the inserter instrument can be separated from the implant 100. This process can be repeated to insert multiple implants, reposition implants, remove implants, or combinations thereof.

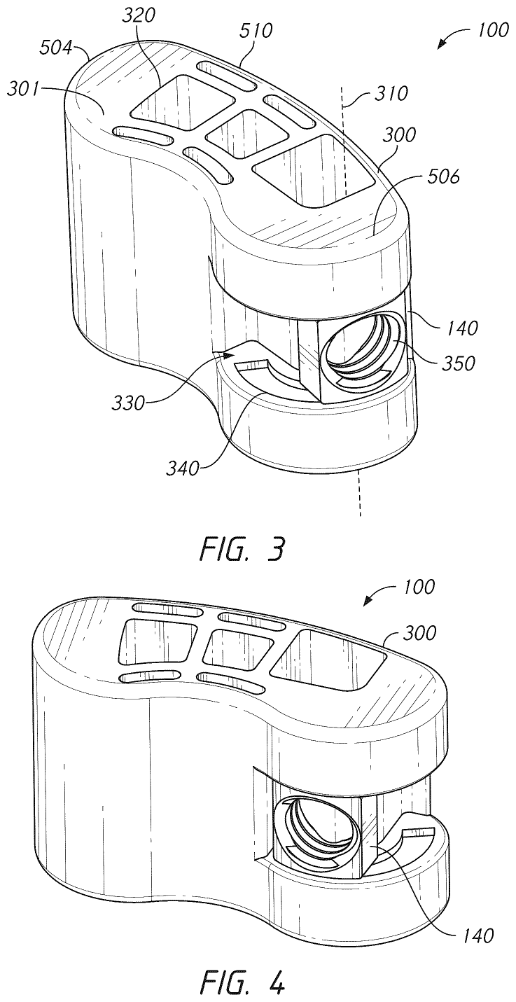

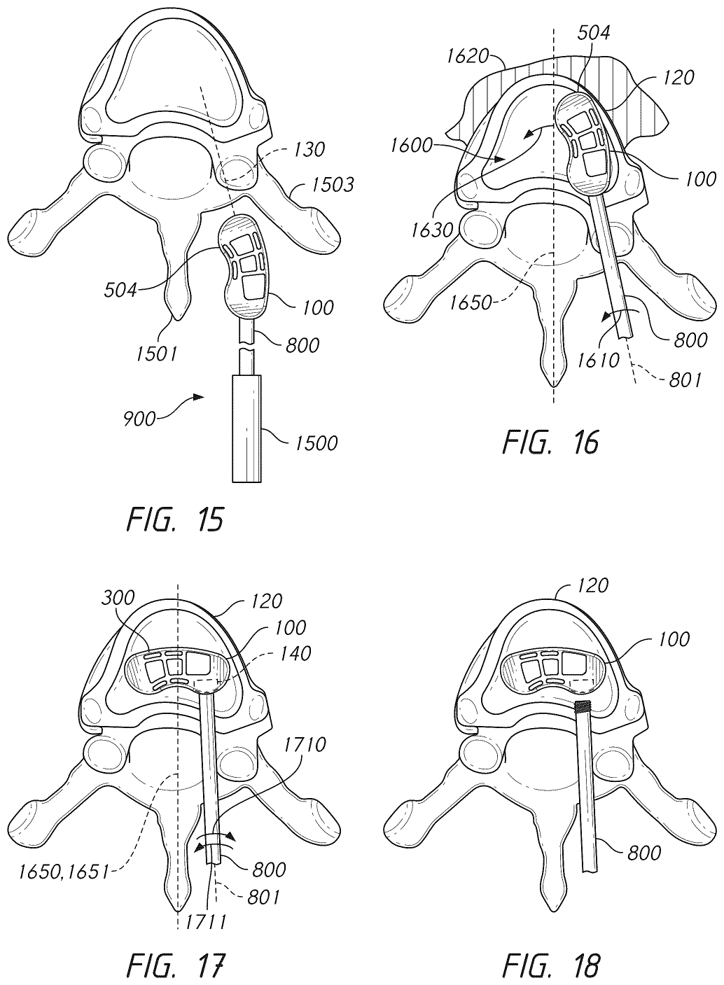

Referring now to FIGS. 1 and 2, the implant 100 can include an instrument coupler 140 (shown in phantom line in FIG. 2) configured to couple to an inserter instrument. When the implant 100 is locked (e.g., locked via engagement with the insert instrument), a main body of the implant 100 can be rigidly attached to the inserter instrument so that the in-line implant and inserter instrument (not shown in FIG. 1) are inserted together. This allows the implant 100 to be delivered along a generally straight transforaminal delivery path 130 (FIG. 3) via, for example, a cannula. The inserter instrument can be operated to unlock the implant 100, which can then rotate (e.g., rotate laterally, anteriorly, etc.) towards the midsagittal plane 138 of the patient and then anteriorly to a transverse rotation. When the implant 100 is unlocked (e.g., unlocked using the insert instrument), the main body of the implant 100 can move relative to the inserter instrument. In some implantation procedures, the implant 100 can be advanced against tissue surrounding the disc space such that the tissue causes the implant 100 to gradually move until the implant 100 is positioned at a desired position (e.g., a transverse position along the vertebral end plate 150) The inserter instrument can remain generally positioned along the transforaminal path 130 while the implant 100 is moved laterally away from the delivery path 130, as discussed in connection with FIGS. 15-18. Example implants can have component(s) that move relative to the insert instrument along a reference plane generally parallel to a plane (e.g., a transverse plane of the patient, a transverse plane of the disc space, etc.), about an axis of rotation (e.g., a superior-inferior axis of rotation or about another axis of rotation or relative to another plane), or the like.

The implants 100, 110 of FIGS. 1 and 2 can be custom, patient-specific implants designed to, for example, reshape the spine, improve spine stability, provide spinal alignment, replace or repair damaged disc, or the like. Fasteners, plates, rods, screws, and/or other features or components can be used in combination with the implants 100, 110. The number, configuration, and positions of implants can be selected to achieve a planned treatment (e.g., spinal correction, target pathology, or the like). The implants can be non-expandable or expandable devices (e.g., interbody cage fusion devices, articulating artificial discs, etc.) that can be filled with material (e.g., bone graft, growth factor, etc.) that promotes growth through the devices to fuse adjacent vertebrae. The description of the implants of FIGS. 3-14 and 20-23 applies equally to the implant 110 of FIG. 1 unless indicated otherwise. Example implants are discussed in connection with FIGS. 3-14 and 20-23, and example implantation procedures are discussed in connection with FIGS. 15-19B.

FIG. 3 is an isometric view of the implant 100 in a delivery configuration in accordance with an embodiment of the present technology. FIG. 4 is an isometric view of the implant 100 in an implantation configuration. Referring now to FIG. 3, the implant 100 can include a fusion or main body 300 (“fusion body 300”) and the instrument coupler 140. The fusion body 300 is configured to contact vertebral end plates of adjacent vertebrae. In some embodiments, the fusion body 300 is a one-piece unitary cage with openings or cavities 320 (one identified in FIG. 3) configured to receive material (e.g., bone graft material) for promoting fusion. The number, configuration, and/or location of the cavities 320 can be selected based on, for example, load-bearing capabilities of the fusion body 300, desired volume for holding material, characteristics of the fusion process, or the like.

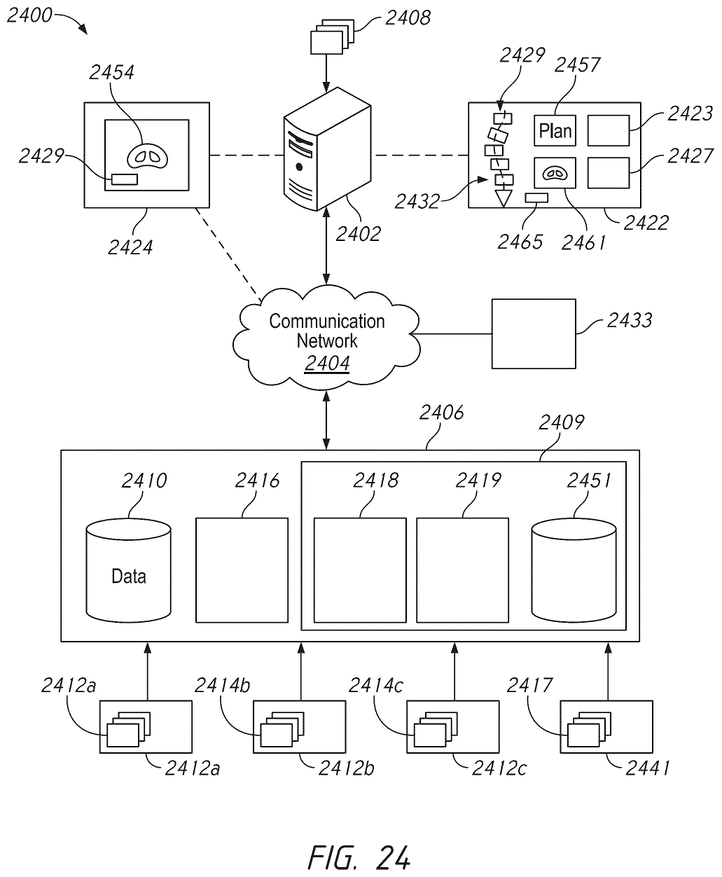

The fusion body 300 can be made, in whole or in part, of metal, plastic and other materials. Example materials and manufacturing techniques are discussed in connection with FIG. 24. FIG. 3 shows the fusion body 300 including a distal end 504, a proximal end 506, and an elongated body 510 extending therebetween. The distal and proximal ends 504, 506 can have atraumatic configurations to inhibit, limit, or substantially prevent injury to tissue. For example, the distal end 504 can have a generally rounded or curved configuration for sliding along tissue. This allows the distal end 504 to atraumatically slide along tissue (e.g., tissue surrounding and/or adjacent to the disc space). The shape and configuration of the fusion body 300 can be selected based on the delivery technique, characteristics of the implantation site, loading configuration, or the like.

The fusion body 300, as viewed from above (see FIGS. 8A and 8B), can be generally C-shape, crescent-shape, or other shape and can be made, in whole or in part, of metal, plastic, composites, and biocompatible materials and may be designed occupy the entire disc space or a portion thereof (e.g., a front or anterior region, a back or posterior region, etc.). The fusion body 300 can include patient-specific contact surfaces 301 (one identified in FIG. 3) designed to match the contours of patient surfaces (e.g., patient's vertebral surfaces, bone surfaces, etc.). In some embodiments, the contact surfaces 301 can generally be planar, wavy, textured, or have another configuration selected based on the procedure. For example, the contact surfaces 301 may be non-patient-specific surfaces that can be used to treat a wide range of patients.

The fusion body 300 can also include a coupler-receiving window or opening 330 (“coupler-receiving window 330”) and one or more tracks 340. The instrument coupler 140 can slide along tracks 340 move between multiple positions, such as a first or initial position (FIG. 3), one or more intermediate positions, and/or a deployed or implantation position (FIG. 4). Referring to FIG. 3, the instrument coupler 140 can be rotatable about an axis of rotation 310. The position and orientation of the axis of the rotation of 310 can be selected based on the targeted movement of the fusion body 300 relative to an inserter instrument. For example, the access rotation 310 can be generally perpendicular to a transverse plane (e.g., transverse plane 604, FIG. 6) or another reference plane defined by the fusion body 300.

The inserter coupler 104 can cooperate with an inserter instrument to block the implant 100. For example, when an inserter instrument extends through an instrument-receiving feature 350 and contacts the fusion body 300, the instrument coupler 140 can be held firmly against to the fusion body 300. The inserter instrument can be torqued to cause the inserter instrument to be moved towards or away from the fusion body 300 to increase or decrease the pressure applied by the inserter instrument to the fusion body. In this manner, the locking force can be increased or decreased by rotating the inserter instrument about its longitudinal axis. The inserter can be torqued to move the inserter instrument away from the fusion body 300, thereby locking the implant 100. When the implant 100 is unlocked (e.g., when the inserter instrument is spaced apart from the fusion body 300), the instrument coupler 140 is movable from the initial position (FIG. 3) to the implant position (FIG. 4), thereby rotating the fusion body 300 relative to the inserter instrument. Example reconfiguring and positioning of the implant 100 are discussed in connection with FIGS. 15-18.

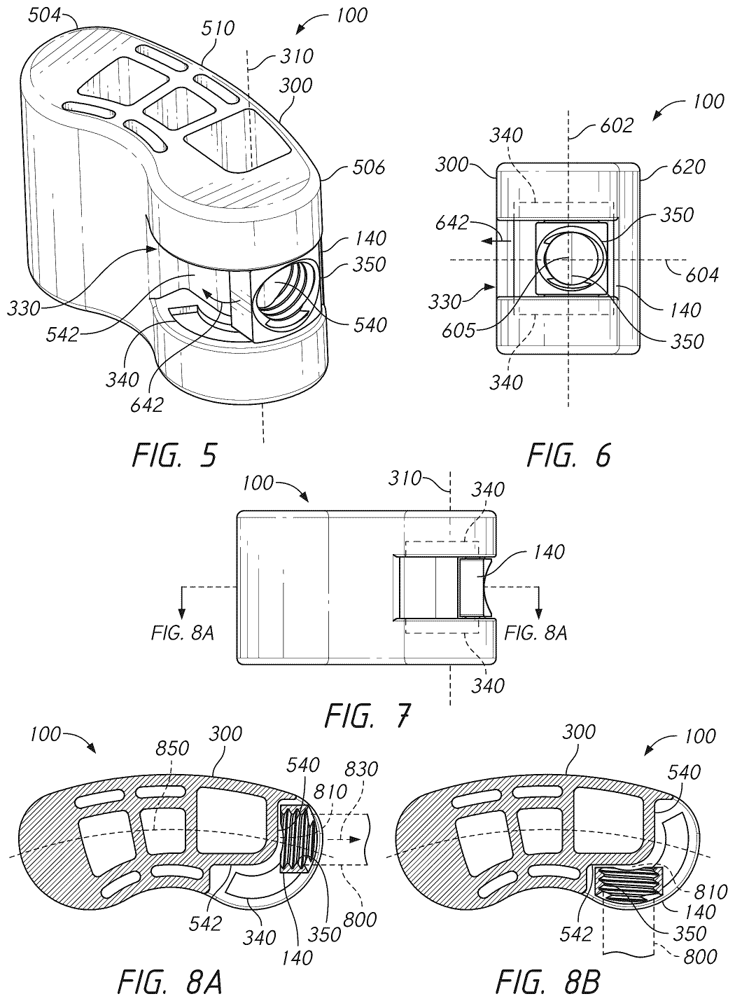

FIGS. 5-7 show details of the implant 100. Referring now to FIGS. 5 and 6, the instrument coupler 140 can include an instrument-receiving feature 350 positionable generally along a frontal plane 602 (FIG. 6) and/or a transverse plane 604 (FIG. 6) of the implant 100. The instrument-receiving feature 350 can be the form of an internally threaded (e.g., M4, M5, M4-locking threads, etc.) throughhole that provides access to the fusion body 300. For example, the instrument-receiving feature 350 can generally align along the frontal plane 602 and/or the transverse plane 604. In some embodiments, an intersection 605 of the frontal plane 602 and transverse plane 604 can pass through the instrument receiving feature 350, as shown in FIG. 6. In certain embodiments, the intersection 605 can be generally centered with respect to the instrument receiving feature 350. The instrument coupler 140 can travel along the track 340, as indicated by arrow 642, to move across a portion of or the entire the window 330. In some embodiments, the instrument coupler 140 moves along the outer boundary of the proximal end 620.

FIG. 7 is a front view of the implant 100 in accordance with an embodiment of the technology. Referring now to FIGS. 5 and 7, the tracks 340 can have arcuate shapes to guide movement of the instrument coupler 140 about the longitudinal axis 310 (340). Referring now to FIG. 8A, the fusion body 300 can include first and second locking faces 540, 542 positioned relative to one another to define lockable positions of the instrument coupler 140. For example, the first locking face 540 extends across the instrument-receiving feature 350 when the instrument coupler 140 is in the initial position. The second locking face 542 extends across the instrument receiving feature 350 when the instrument coupler 140 is in the implant position (FIG. 8B). The number and/or positions of the locking faces can be selected based on the desired number of lockable configurations of the implant 100.

FIG. 8A is a cross-sectional view of the implant 100 taken physically along line 8A-8A of FIG. 7. An inserter instrument 800 (shown in phantom line) can contact the surface 540 so that forces (e.g., distal forces) are applied directly to the main body 300 to minimize or limit damage to the instrument coupler 140 if a user hammers (e.g., using a mallet) on the inserter instrument 800. The faces 540, 542 can be generally flat to provide relatively large surface areas to which forces can be applied. The surfaces 540, 542 can also be generally orthogonal to one another to allow the instrument coupler 140 to be rotated about 80, 90, or 100 degrees relative to the axis 310. The number, sizes, and/or configurations of the faces 540, 542 can be selected based on number of pre-set implant configurations.

The locked implant 100 is fixedly coupled to the instrument 800 to allow positioning within the patient. To lock the implant in, flat distal surface 810 of the insertion instrument 800 can lie flat along the illustrated planar face 540. A user can rotate the inserter instrument 800 clockwise or counterclockwise to increase or decrease, respectively, the pressure applied to the face 540. The instrument 800 can also be rotated counterclockwise to move the instrument 800 away from the face 540. After the inserter instrument 800 is spaced apart from the face 540, the instrument coupler 140 can be slid along the track 340 from a mid-plane or centerline 850 of the implant 100. The inserter instrument 800 can remain coupled to the instrument coupler 140 during this movement. A user can then rotate the inserter instrument 800 clockwise to move the distal surface 810 against the second face 542, as shown in FIG. 8B, thereby relocking the implant 100. The inserter instrument 800 (illustrated in phantom line) can bear against the surface 542 to relock the implant 100.

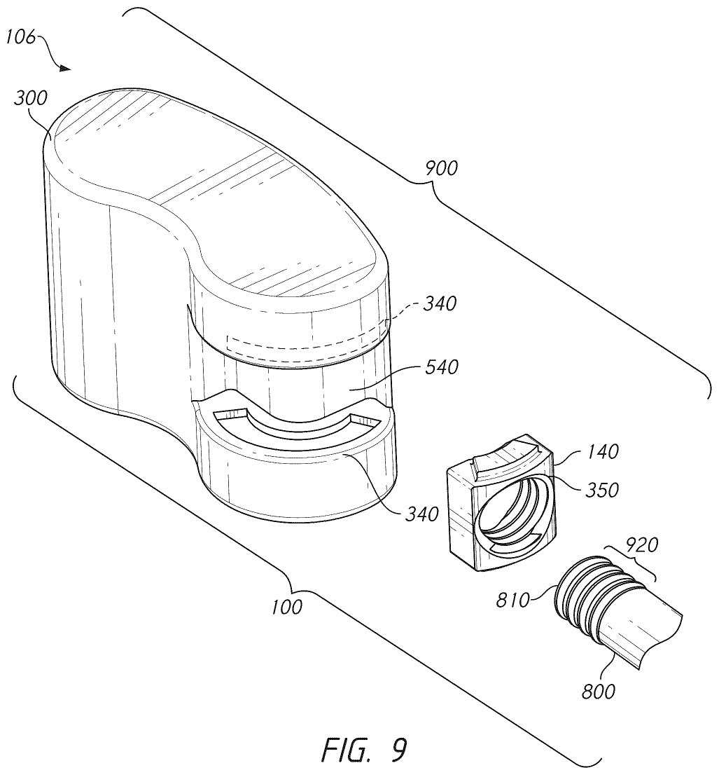

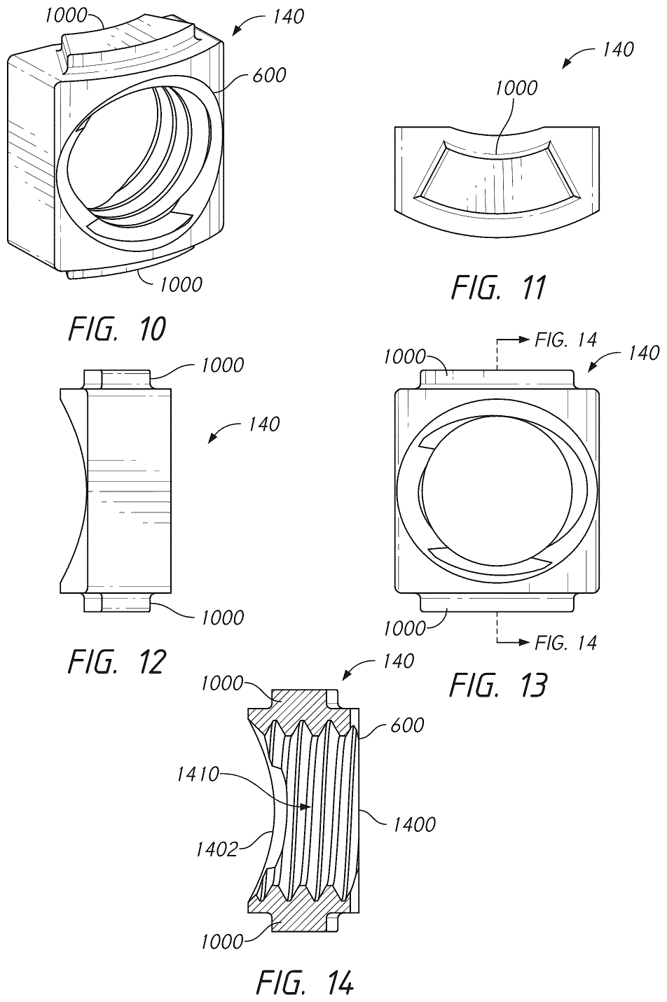

FIG. 9 is an exploded isometric view of a surgical system 900 in accordance with an embodiment of the present technology. The surgical system 900 can include the implant 100 and the inserter instrument 800. The inserter instrument 800 includes a distal portion 920 with external threads configured to engage the instrument-receiving feature 350. To assemble the surgical system 900, the distal portion 920 can be inserted into the instrument-receiving feature 350. The inserter instrument 800 can be rotated clockwise about its longitudinal access 801 to advance the inserter instrument 800 distally towards the wall 540 of the implant body 300. The user can torque the inserter instrument 800 until the face 810 of the inserter instrument 800 physically contacts the implant body 300 to apply desired pressure for locking the implant 100 in the desired configuration. The surgical system 900 can be assembled prior to inserting the implant 100 into the patient. The user can select the length of the inserter instrument 800 based on the depth of the implantation site. Details of the instrument coupler 140 are discussed in connection with FIGS. 10-14. The number of components, configuration, and/or operation of the surgical system 900 can be selected based on the surgical procedure to be performed.

FIGS. 10-14 show the instrument coupler 140 in accordance with an embodiment of the present technology. The instrument coupler 140 can include guides 1000 configured (e.g., curved or arcuate configurations) to generally match the configurations of the tracks 340 (FIG. 9). As shown in FIG. 14, the receiving-feature 350 can include a proximal opening 1400, a distal opening 1402, and a bore 1410 extending therebetween. The internally threaded surface can extend between the proximal opening 1400 and the distal opening 1402. The length and size of the receiving feature at 350 can be selected based on the forces to be applied to the coupler 140 during operation. For example, the length of the receiving feature 350 can be increased or decreased to allow for increased or decreased pressures to be generated and applied to the implant body 300. The features and configuration of the instrument coupler 140 can be selected based on the desired movement of the instrument coupler 140 during the surgical procedure.

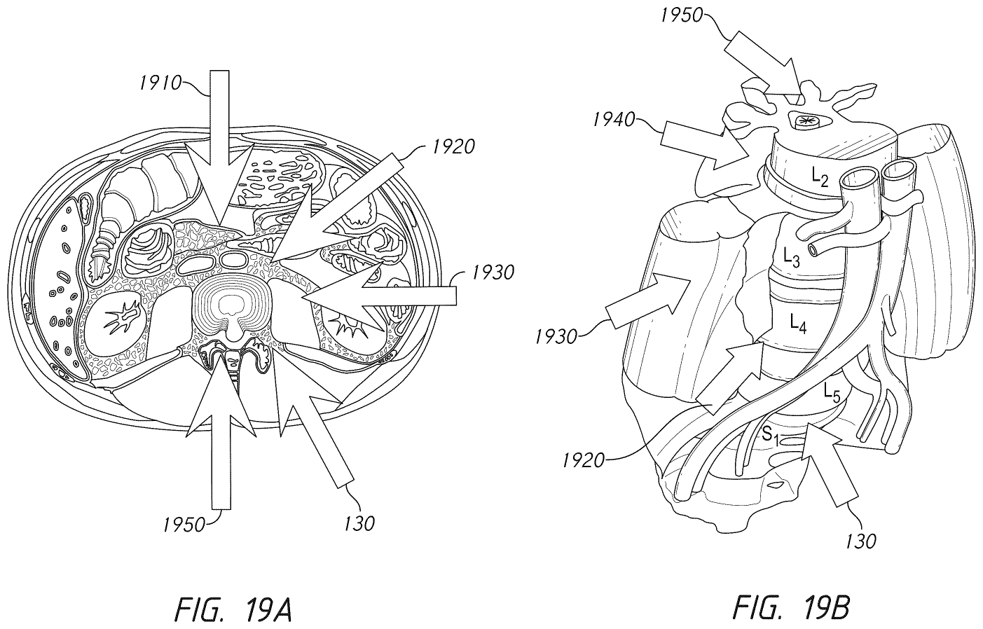

FIGS. 15-18 show a method for implanting the implant 100 in accordance with an embodiment of the present technology. The design of the surgical system 900 allows for in-vivo reconfiguring (e.g., locking, unlocking, expanding, collapsing, rotating, etc.) of the implant 100. In general, the implant 100 can be selectively locked and/or unlocked to manipulate the implant 100 without translating the inserter instrument 800. The implant 100 can be designed to provide patient-specific angular rotation limits (e.g., via pivoting instrument couplers, sliding instrument coupler, etc.). During delivery towards the implantation site, the locked implant 100 can be rigidly coupled to the inserter instrument 800. The inserter instrument 800 can then be used to unlock the implant 100 such that the unlocked implant 100 is movable relative to the instrument 800. A user can use the instrument 800 to move the implant 100 along the disc space. After the implant 100 is at a desired location, the instrument 800 can be detached from the implant 100. Some example delivery techniques and steps are discussed in connection with FIGS. 15-18 and additional delivery techniques and steps are discussed in connection with FIGS. 19A and 19B.

Referring now to FIG. 15, the implant 100 can be rigidly coupled to the inserter instrument 800. The system 900 can have a generally linear configuration to allow delivery of the implant 100 along a relatively small diameter cannula, port, or delivery path. In example transforaminal delivery techniques, a user can prepare the intervertebral disc space by removing tissue (e.g., soft tissue, bony tissue, etc.). In addition, bone spurs can be removed, and cartilage can be removed for decompression. For example, a facet joint, which couples adjacent vertebral bodies, can be removed to access the intervertebral disc space. Once access is gained, the user can remove disc material from the intervertebral space and can also roughen the exposed surfaces of the adjacent vertebrae to prepare the implantation site.

The user can assemble the surgical system 900 and lock the implant 100. The user can grip a handle 1500 of the inserter instrument 800 to advance the implant 100 distally along the transforaminal delivery path 130. The transforaminal delivery path 130 (as viewed from above) can be positioned between the spinous process 1501 and the adjacent transverse process 1503 and can serve as a relatively linear path along which the implant 100 is advanced distally using the inserter instrument 800. In some embodiments, the inserter instrument 800 can have a unitary one-piece construction to provide for reliable operation. This reduces or eliminates potential risk of movable instrument components breaking or failing. Additionally, relatively large forces can be applied to the implant 100 while limiting, minimizing, or preventing bending or buckling of the inserter instrument 800. The main body of implant 100 rotates away from an imaginary plane extending from the inserter instrument 800 and/or transforaminal delivery path 130. The angle of rotation can be relatively oblique to the imaginary plane extending from the inserter instrument 800 and/or transforaminal delivery path 130.

The instrument coupler 140 can couple the instrument 800 to the implant 100 such that the flat distal end of instrument 800 can lie generally flush to the planar face of implant 100. The instrument coupler 140 can be configured to maintain high contact between the instrument 800 and the implant 100 to reduce or limit the one or more forces applied to the main body 300 of implant 100. In addition, the flush configuration of the coupled instruments can prevent undesired lateral movement of the implant 100 as the coupler 140 slides along the curvature of the tracks of implant 100 from the initial position to the deployed position.

FIG. 16 shows the implant 100 after it has been positioned in an intervertebral disc space 1600. The inserter instrument 800 can then be used to unlock implant 100 which can be held captive between adjacent vertebral bodies. For example, the user can rotate the instrument 800 in a counterclockwise direction (indicated by arrow 1610) about its long axis to unlock the implant 100. The user can advance the unlocked implant 100 distally to cause the distal end 504 to slide along tissue 1620. The tissue 1620 can guide movement of the implant 100 in the lateral direction, as indicated by arrow 1630. In this manner, the distal end 504 can be moved away from the longitudinal axis 801 of the instrument 800, the transforaminal delivery path 130, or the like.

The instrument 800 can be generally positioned along, or parallel to, the transforaminal delivery path 130 while the implant 100 is rotated to move across a midsagittal plane 1650 of the patient or midline of the disc space. In some procedures, the entire instrument 800 can remain on one side of the midsagittal plane 1650 while the implant 100 is moved toward and across the midsagittal plane 1650. In some embodiments, the inserter instrument 800 can have unitary one-piece construction to provide for reliable operation. This reduces or eliminates potential risk of movable instrument components breaking or failing. Additionally, relatively large forces can be applied to the implant 100 while limiting, minimizing, or preventing bending or buckling of the inserter instrument 800. The inserter instrument 800 can be displaced to translate or linearly move the implant 100 along the vertebral body 120. The adjacent vertebral bodies can inhibit, limit, or substantially prevent rotation of the implant 100 in the superior to inferior direction. This allows rotation of the inserter instrument 800 about its longitudinal axis 801 to increase or decrease the pressure applied by the instrument 800 to the implant body 300.

The coupler 140 can slide along the curvature of the tracks of implant 100 at varying angular ranges (e.g., at least 30 degrees, 50 degrees, 80 degrees, 90 degrees, 100 degrees, etc.). The length and curvature of the tracks of implant 100 can differ based on the patient's anatomy (e.g., intervertebral space), target implant position for the implant 100, etc.

The instrument 800 can be a unitary inserter instrument, such that the instrument 800 comprises no moving parts and can be locked and/or unlocked without additional screws or moving components. The instrument 800 can be used to rotate the main body 300 of the implant 100 across the vertebral body 120 or the midline of the patient's disc space. A user can apply relatively large forces (e.g., via a hammering, hand pressure, manual pulling, etc.) directly to the main body 300 of the implant 100. The torque and/or moment that moves the main body 300 of the implant 100 can be proportional to the one or more forces applied directly to the main body 300. The instrument 800 can be, for example, moved side-to-side, rotated, pushed distally, and/or pulled proximally to position or remove the implant 100.

FIG. 17 shows the implant 100 extending laterally across the vertebral body 120. In some procedures, a midsagittal plane 1651 of the implant 100 can be parallel to or lying along the midsagittal plane 1650 of the patient. Visualization techniques can be used to evaluate the position of the implant 100. Example visualization techniques include, for example, fluoroscopy, x-rays, direct visualization, and/or viewing via optical cameras. The instrument 800 can be rotated clockwise direction (indicated by arrow 1710) about its longitudinal axis 1610 to relock the implant 100. FIG. 18 shows the instrument 800 after it has been separated and moved apart from the implant 100. After the implant 100 is at a target position, a user can rotate the instrument 800 in a counterclockwise direction (indicated by arrow 1711) to decouple the instrument 800 from the implant 100. An optional locking bolt or member can be inserted into the instrument coupler 140 to limit or prevent movement (e.g., post-implantation movement) of the instrument coupler 140.

The instrument 800 can be reattached to the implant 100 to reposition or remove the implant 100. To reposition the implant 100, the instrument 800 can be reattached to the instrument coupler 140. The instrument 800 can then be used to push or pull the implant 100 to adjust the position along the vertebral endplate 120. To remove the implant 100, the instrument 800 can be pulled distally. The implant 100 can be in an unlocked state to allow rotation relative to the instrument 800 to minimize, limit, or substantially prevent damage to surrounding tissue. As the instrument 800 is pulled proximally, the implant 100 and instrument 800 can assume a generally linear configuration for removal along a narrow path. The implant 100 can be locked or unlocked any number of times to assist with removal, repositioning, or the like.

A variety of different surgical paths can be used to deliver selectively lockable implants. Referring now to FIGS. 19A and 19B, implants can be delivered via different surgical approaches. Example approaches include, without limitation, anterior approaches, oblique approaches, lateral approaches, transforaminal approaches, and/or posterior approaches. For example, the implants can be delivered along an anterior lateral lumbar interbody fusion (ALIF) path 1910, an oblique lumbar interbody fusion (OLIF) path 1920, a lateral lumbar interbody fusion (LLIF) or extreme lateral lumbar interbody fusion (XLIF) path 1930, a transforaminal lumbar interbody fusion (TLIF) path 130, and a posterior lumbar interbody fusion (PLIF) path 1950. These paths can also be used to perform other procedures disclosed herein. For example, one or more of the paths 130, 1910, 1920, 1930, 1950 can be selected for endoscopic approaches to perform a wider array of spine procedures. In some embodiments, a user inputs a planned surgical approach, and a planned delivery path (e.g., one of paths 130, 1910, 1920, 1930, 1950) can be selected for the planned surgical approach. The planned delivery path can be generated based on, for example, one or more simulations, patient images, models, user input, etc. In some embodiments, a system can analyze one or more implant designs to generate planned surgical approaches and planned delivery paths (e.g., one of paths 130, 1910, 1920, 1930, 1950) for the one or more implant designs.

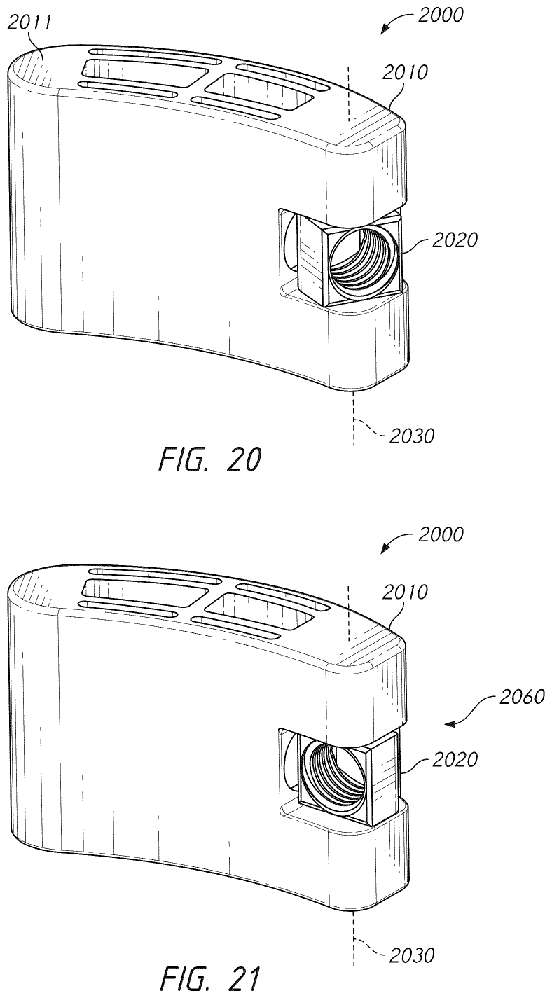

Surgical instruments can be used to remove tissue to form access paths, working space(s), and/or modified implantation sites inside the patient. In some example TLIF procedures, the transforaminal path 130 may be employed to implant a single small implant (e.g., implant 100) at the intervertebral space. In some example PLIF procedures, two implants (e.g., implants 2000 of FIGS. 20 and 21) can be delivered along the posterior path 1910 and implanted at the intervertebral space. The two interbody spacers can cooperate to keep the vertebral bodies at the desired spacing and may be more slender than the TLIF spacer. Additionally, multiple interbody spacers can provide lordotic correction by providing support at different heights. In some example LLIF procedure, a single, relatively large implant can be delivered along the lateral path 1930 and implanted to provide asymmetrical support. In some example ALIF procedure, an asymmetric interbody spacer can be delivered along the anterior path 1910 to provide support consistent with lordosis at that portion of the spine. Lateral approaches, transforaminal approaches, and anterior approaches can be used to access the cervical spine, thoracic spine, etc. The number of instruments, configurations of instruments, implants, and/or surgical techniques can be selected based on the condition to be treated.

The configuration of implants can be selected based on a surgical path. The surgical path selected can be used to access varying portions of the spine (e.g., the cervical spine, thoracic spine, lumbar spine, sacrum, etc.). For example, in the some example TLIF procedures, the transforaminal path 130 can be employed to deliver the single small expandable or non-expandable interbody spacer at the intervertebral space. For delivery, the single small expandable or non-expandable interbody implant can comprise a track that is greater in length and curvature than track 340 of FIG. 3, allowing the lockable implant to rotate at an angle (e.g., 90 degrees, 100 degrees, 110 degrees, 120 degrees, etc.) to position the spacer. In some example LLIF procedures, the lateral path 1930 can be employed to deliver the single, large interbody spacer. For delivery, the single, large interbody spacer can comprise a track that is shorter in length and less curved than track 340 of FIG. 3, allowing the lockable implant to rotate at a smaller angle, for example, 10 degrees. In some example ALIF procedures, the anterior path 1910 can be employed to deliver an asymmetric interbody implant configured with a track that has a length and curvature that allows for increased rotation of the lockable implant, for example, more than 100, 145, 180 degrees. Based on the surgical path employed, a user can select the number of implants, implant shape, and/or motion of implantation used. Motion of implantations can be based on the amount of side-to-side movement, rotations, pushing distally, pulling distally, or the like required to position the implant.

FIGS. 20 and 21 are isometric views of an implant 2000 including a fusion body 2010 and a pivoting instrument coupler 2020 in accordance with an embodiment of technology. The description of the implant 100 of FIGS. 1-19B applies equally to the implant 2000 unless indicated otherwise. Referring now to FIGS. 20 and 21, the instrument coupler 2020 is configured to pivot or swivel about an axis of rotation 2030 extending in a superior-inferior direction (relative to the fusion body 2010, patient, etc.), generally perpendicular to face 2011. In some embodiments, the axis of rotation 2030 is generally perpendicular to a transverse plane of the implant 2000. The instrument coupler 2020 can include one or more pins, hinges, or pivot elements engaging the fusion body 2010 to allow the instrument coupler 2020 to rotate from a delivery position (FIG. 20) to an implant position (FIG. 21).

The implant 2000 of FIGS. 20 and 21 can include a relatively slender (as viewed from the side) and tall fusion body 2010. The pivoting instrument coupler 2020 can be advantageous over one or more-track designs of FIG. 3. For example, multiple implants 2000 can be individually implanted to distribute loading along multiple regions (e.g., spaced apart regions) of the vertebral body (e.g., a front or anterior region, a back or posterior region, etc.) rather than just at one continuous region of the vertebral bodies. As the multiple implants are positioned to provide desired load support, the instrument coupler 2020 can pivot about the axis of rotation 2030 and can lock the implant position (FIG. 21) into place. An optional locking bolt or member can be inserted into the instrument coupler 2020 to limit or prevent movement (e.g., post-implantation) of the instrument coupler 2020. The number, configuration, and/or positions of the implants 2000 can be selected based on the characteristics of the spine, targeted spinal correction, etc.

In some procedures, multiple implants in combination with anchoring screws and/or rods can provide additional support between the vertebrae and can restore the height of the intervertebral space. Anchoring screws and/or rods can be used to distribute the load dependent on the level of fusion necessary for the condition being treated. Implant configurations can include expandable and/or non-expandable cages of varying shapes (e.g., C-shaped, crescent shaped, straight, etc.) that are best fit (e.g., meets a best fit score threshold, physician approval, etc.) to stabilize a portion of the spine for the condition being treated. When the instrument coupler 2020 is in the delivery position (FIG. 20), the fusion body 2010 can move relative to an inserter instrument used to position the implant (e.g., at the anterior region, posterior region, etc.). The rods can be approach-specific designed rods, patient specific rods, etc.

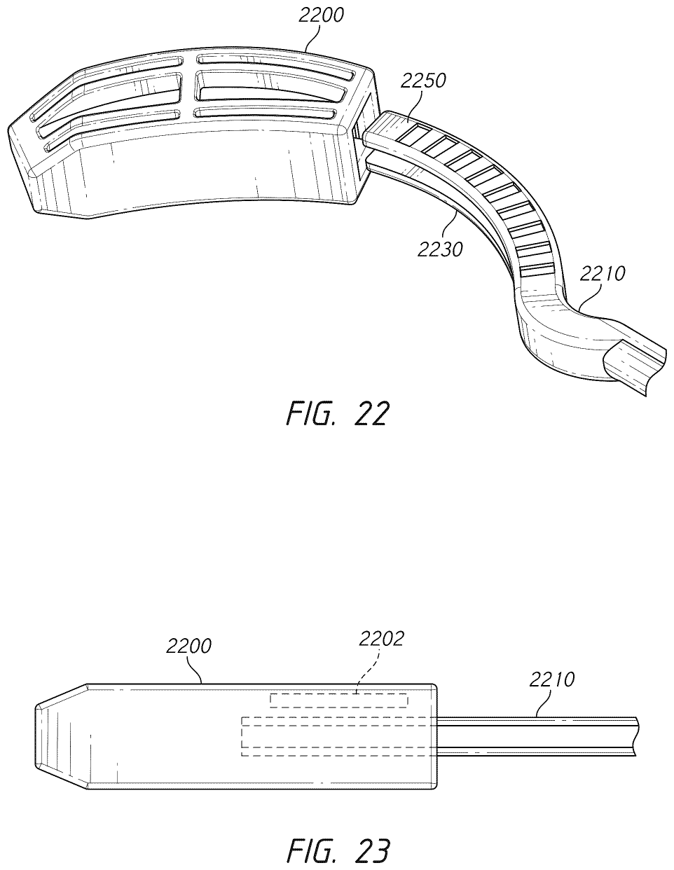

FIGS. 22 and 23 are isometric views of a ratcheting implant 2200 and an inserter instrument 2210 in accordance with an embodiment of the technology. The description of the implants 100, 2000 applies equally the implant 2200 unless indicated otherwise. The implant 2200 includes a ratcheting instrument coupler 2202 configured to engage the instrument 2210 such that the ratcheting implant 2200 is movable along an arcuate path along a curved portion 2230 of the inserter instrument 2210. The curved portion 2230 can include ratcheting features 2250 (one identified in FIG. 22).

The ratcheting instrument coupler 2202 of implant 2200 can be driven forward along the curved portion 2230 of inserter instrument 2210 by one or more forces. This allows radial movement of the implant 2200 while the instrument may be generally stationary. For example, the implant 2200 can be distally driven along the curved portion of 2230 by a mechanical force output by a user (e.g., via hand pressure, manual pushing, etc.). In another example, the implant 2200 can be distally driven along the curved portion 2230 by a pressurized fluid (e.g., saline, water, etc.). In another example, the implant 2200 can be distally driven along the curved portion 2230 by an energy field. More specifically, the inserter instrument 2210 can include one or more electrodes (e.g., electrodes along the curved portion 2230) that generate electromagnetic fields to advance the implant 2200. The same driving forces for moving the implant 2200 can be employed for unlocking/locking (e.g. unlocking the implant 2200 from the ratcheting instrument coupler 2202, locking the implant 2200 to the ratcheting instrument coupler 2202, etc.).

The patient-specific implant can be any of the implants described herein or in any patent references incorporated by reference herein. For example, the patient-specific implant can include one or more of screws (e.g., bone screws, spinal screws, pedicle screws, facet screws), interbody implant devices (e.g., intervertebral implants), cages, plates, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include articulation, implant shape, size, material, and/or effective stiffness (e.g., lattice density, number of struts, location of struts, etc.). Examples of patient-specific implants that can be designed at block 314 are described in U.S. application Ser. Nos. 16/048,167, 16/242,877, 16/207,116, 16/352,699, 16/383,215, 16/569,494, 16/699,447, 16/735,222, 16/987,113, 16/990,810, 17/085,564, 17/100,396, 17/342,329, 17/518,524, 17/531,417, 17/835,777, 17/851,487, 17/867,621, and 17/842,242, each of which is incorporated by reference herein in its entirety.

FIG. 24 is a network connection diagram illustrating a system for providing patient-specific medical care and configured in accordance with select embodiments of the present technology. The present technology also provides systems and methods for designing and manufacturing patient-specific implants, such as any of the patient-specific vertebral implants described herein. The system can be used to manufacture the implants (implants 100, 2000, 2100, 2600, 3200, 3800, 4500, 4900, 5500) with printed bodies, instrument couplers, and/or other components (e.g., instruments). In some embodiments, implants are printed in-place and do not require any post-print assembly operations. In some embodiments, components of the implants can be individually printed (e.g., 3D-printed Titanium cage) and then subsequently assembled. The rotational limits of implants may be customized for each patient or may be set as a fixed parameter of the design. The rotational limits can be incorporated into a surgical plan reviewable by a physician. In some embodiments, the sliding thread parts can be nested and not exposed to the patient's disc space, and the cages can incorporate patient-matched endplate contours and other features disclosed herein.

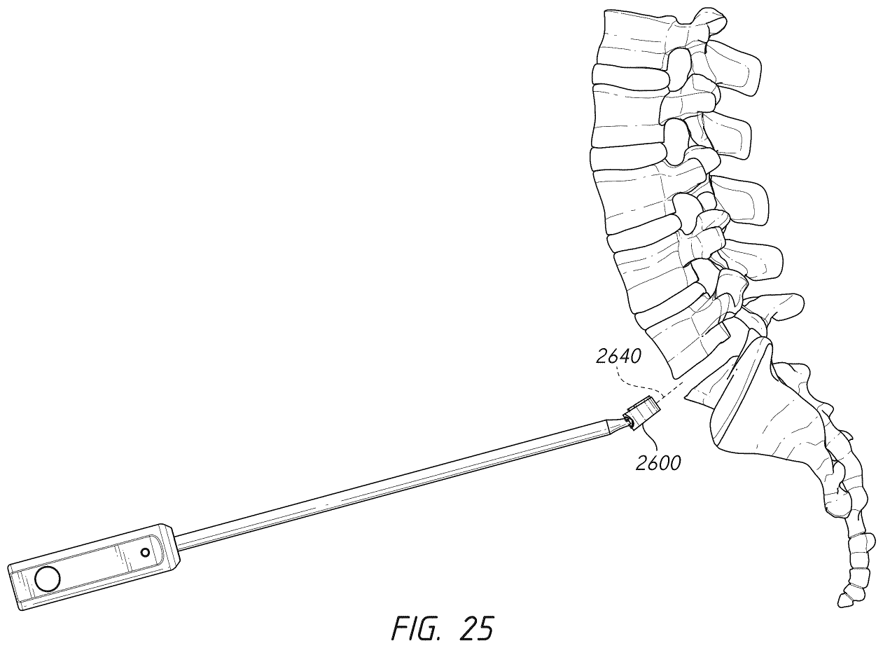

FIG. 24 shows a computing system 2400 that designs implants, instruments, tools, surgical kits, plans, etc. The computing system 2400 can design, for example, one or more implants (e.g., patient-specific implants, rotatable implants, approach-specific implants, combinations thereof) and/or delivery instruments and can optionally generate a patient-specific surgical plan. For example, the computing system 2400 can generate an implant and/or generate a surgical plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof. The surgical plan can include surgical information, surgical plans (e.g., surgical implant procedure, target implant location and/or orientation, etc.), technology recommendations (e.g., device and/or instrument recommendations), and/or medical device designs. For example, the surgical plan can include at least one treatment procedure (e.g., a surgical procedure or intervention) and/or at least one medical device (e.g., an implanted medical device (also referred to herein as an “implant” or “implanted device”) or implant delivery instrument or inserter instrument). In some embodiments, the computing system 2400 determines a planned in-vivo rotation of an implant based on a target anatomical correction and a planned delivery path and designing the implant to move relative to an instrument the planned in-vivo motion such that the implant is moved to a target position while the instrument is positioned along the planned delivery path for the surgical plan. The user can revise the surgical path, surgical plan, and/or in vivo motion and simulate the surgical procedure to evaluate the modified surgical plan. In some embodiments, the computing system 2400 designs surgical approach-specific implants based on, for example, one or more target anatomical corrections, planned delivery paths, and/or delivery instruments. Example surgical approach-specific implants are discussed in connection with FIGS. 25-64. The surgical approach-specific implants can include rotatable/articulating coupling features discussed in connection with FIGS. 1-21, fixed coupling features discussed in connection with FIGS. 22-23 and 25-64, combinations thereof, or the like. In some embodiments, the implants are designed for at least a portion of a planned delivery path to the implantation site.

The computing system 2400 includes a computing device 2402, which can be a user device, such as a smart phone, mobile device, laptop, desktop, personal computer, tablet, phablet, or other such devices known in the art. The computing device 2402 can include one or more processors, and memory storing instructions executable by the one or more processors to perform select methods described herein. The computing device 2402 can be associated with a healthcare provider that is treating the patient. Although FIG. 24 illustrates a single computing device 2402, in alternative embodiments, the computing device 2402 can instead be implemented as a computing system encompassing a plurality of computing devices, such that the operations described herein with respect to the computing device 2402 can instead be performed by the computing system and/or the plurality of computing devices.

The computing device 2402 is configured to receive a patient data set 2408 associated with a patient to be treated. The patient data set 2408 can include data representative of the patient's condition, anatomy, pathology, symptoms, medical history, preferences, and/or any other information or parameters relevant to the patient. For example, the patient data set 2408 can include surgical intervention data, treatment outcome data, progress data (e.g., physician notes), patient feedback (e.g., feedback acquired using quality of life questionnaires, surveys), clinical data, patient information (e.g., demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like. In some embodiments, the patient data set 2408 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine.

In some embodiments, the patient data set 2408 includes externally generated data, third-party data collected from external sources (e.g., remote servers, databases, websites, journals, articles, platforms, etc.), databases, software program platforms, navigation systems, treatment planning platforms, or combinations thereof. The databases can store, for example, design footprints, machine learning modules, algorithms, models (e.g., anatomical models), etc. The computing device 2402 can communicate with one or more remote software program platforms. For example, the computing device 2402 can authenticate and receive data from authenticated software programs or platforms and use the received data to generate treatment plans. In some embodiments, the computing device 2402 can be periodically or continuously synchronized with a third-party data source. The third-party data source can include a computer-aided design (CAD) platform or application that updates models, and the updated models can be automatically sent to, or retrieved by, the computing device 2402. The computing device 2402 can validate the third-party model for one or more of treatment planning, navigation, manufacturing, or the like. For example, the third-party model can be validated by, for example, authenticating the third-party data source, translating the third-party model into a readable digital format, modifying the third-party virtual model for importation into an existing model, confirming that the imported third-party virtual model is compatible with other model elements, and/or importing all or a portion of the third-party virtual model and associated data (e.g., spinopelvic metrics or parameters). The imported third-party model can include one or more two-dimensional (2D) or three-dimensional (3D) models of one or more anatomical elements, implants, navigation equipment, and/or instruments.

The computing device 2402 can communicate with the third-party data source such that the third-party data source modifies, replaces, or adjusts the data. In some embodiments, the computing device 2402 can be periodically or continuously synchronized with third-party treatment planning software, CAD software, navigation software, user device (e.g., smartphone or tablet capturing images of surgery site), robotic surgery system, or the like. In some embodiments, the synchronization can be performed based on one or more synchronization triggers, including modification of data or event (e.g., publication of article(s), planned manufacturing of implant, start of surgery, etc.). In some embodiments, a user can set a schedule (e.g., daily, weekly, monthly, etc.) for synchronization using a mobile application.

The computing device 2402 can combine anatomical models from multiple third-party data sources to generate a multi-source anatomical model. The computing device 2402 can analyze, score, rank, and/or modify the multi-source anatomical model to, for example, match patient images, generate predictions, generate surgical plans, etc. The anatomical models include standard models modifiable (e.g., scaled, sized, or altered to match patient data) based on patient data, patient-specific models with topology matching patient topology, etc. For example, the computing device 2402 can combine one or more standard models of anatomy, patient-specific models, models of implants, and other models to generate multi-source models. The multi-source models can be 2D and 3D location models, topology models, and location-topology models. The location models can represent the anatomical positions of the patient anatomy. Delivery paths can be located between the anatomical elements. The topology models can represent the topology of the patient's anatomy. The location-topology models can represent both the position and the topology of the patient's anatomy. The computing device 2402 and/or user can select metrics and/or parameters for the multi-source models.

The computing device 2402 can combine treatment plans from multiple third-party data sources to generate multi-source treatment plans. In some embodiments, the computing device 2402 can receive multiple treatment plans and integrate the plans together to generate a single multi-source treatment plan. The computing device 2402 can select a set of metrics from a first treatment plan and a second set of metrics from a second treatment plan. A multi-source treatment plan and a virtual anatomical model can be generated to achieve both the first and second sets of metrics. For example, the computing device 2402 can generate a multi-source treatment plan based on the multiple individual treatment plans, such as an anterior fusion plan (e.g., a lumbar fusion plan for implanting one or more cages) and a posterior fusion plan (e.g., a fusion plan for implanting one or more rods). The multi-source treatment plan can simulate outcomes associated with each approach or delivery path, treatment, spine pathology, and the like. The multi-source treatment plan can be synchronized to update information (e.g., metrics, types of visual images, etc.) selected by, for example, a physician, healthcare provider, computing device 2402, machine learning module, or the like. The metrics and/or parameters can be from different models and/or treatment plans. In some embodiments, the multi-source treatment plan can be updated based on modification(s) to a linked third-party source that provided a source treatment plan.

In some embodiments, the computing device 2402 can also be configured to receive a surgical team data set 2410. The surgical team data set 2410 can include data representative of the surgical team that will perform the surgery on the patient. For example, the surgical team data set 2410 can include preferences of the surgical team (e.g., preferred implant techniques, preferred implant instruments/tools, etc.), experience of the surgical team (e.g., past procedures performed by the surgical team), scored outcomes of past procedures performed by the surgical team, or the like. As used herein, the term “surgical team” can refer to a group of healthcare practitioners that work together in an operating room during an implant procedure, or to one or more individual surgeons.

In some embodiments, the computing device 2402 can also be configured to receive a facility or provider data set 2412 (e.g., data sets 2412a-c). The facility data set 2412 can include data representative of the facility at which the patient's surgery will occur. For example, the facility data set 2412 can include preferences of the facility (e.g., preferred implant techniques, preferred implant instruments/tools, etc.), experience of the facility (e.g., past procedures performed at the facility), scored outcomes of past procedures performed at the facility, infrastructure available to assist/perform the surgery (e.g., availability of robotic surgical platforms, as well as the type of “input” required to control the “output” of the robotic surgical platforms), or the like. As used herein, the term “facility” can refer to a single operating room facility, a hospital having multiple operating rooms, and/or a network of hospitals.

The computing device 2402 is operably connected via a communication network 2404 to a server 2406, thus allowing for data transfer between the computing device 2402 and the server 2406. The communication network 2404 may be a wired and/or a wireless network. The communication network 2404, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 2406, which may also be referred to as a “treatment assistance network” or “prescriptive analytics network,” can include one or more computing devices and/or systems. As discussed further herein, the server 2406 can include one or more processors, and memory storing instructions executable by the one or more processors to perform the methods described herein. In some embodiments, the server 2406 is implemented as a distributed “cloud” computing system or facility across any suitable combination of hardware and/or virtual computing resources.

The computing device 2402 and/or the server 2406 can design a patient-specific implant based at least in part on the patient data set 2408, the surgical team data set 2410, and/or the facility data set 2412. For example, the server 2406 may include a treatment planning module 2418 that can design, based off on any of the foregoing data inputs, the implant. In some embodiments, the implant includes a design for a vertebral implant including a cage and a plate, such as any of the implants described with respect to FIGS. 1-23, that are specifically designed to mate with corresponding target anatomical structures. The computing device 2402 and/or the server 2406 can design an interbody device (e.g., a cage), articulation, instrument, a positioning feature (e.g., a plate), a connection mechanism, and/or one or more fastening elements (e.g., a screw). For example, to design the connection mechanism, the target position of the cage can be determined by analyzing images of the patient. The patient spine can be analyzed to identify a suitable target site for the positioning feature. For example, the vertebral bodies can be analyzed to identify regions with suitable geometry and mechanical properties to interface with a locking plate. The computing device 2402 and/or the server 2406 can design the connection mechanism based on a level of movement (e.g., a maximum level of relative movement) between the cage and the locking plate. In spinal fusion procedures, the connection mechanism can be a rigid arm that substantially prevents relative movement between the cage and the plate. In articulating disc procedures, the implant can include an articulating disk, a plate, and one or more flexible or semi rigid connection mechanisms coupling the disk and plate together. The target spatial relationship between the components of the implant can be determined based on the patient data and targeted outcome.

Additional implants include, but are not limited to, screws (e.g., bone screws, spinal screws, pedicle screws, facet screws), other interbody implant devices, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. These implants can include instrument couplers that provide movement of the implant. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.).

The implant can be designed to match the patient's existing anatomy. For example, the implant can be designed such that various surfaces of the implant mate with corresponding surfaces of patient anatomy, as previously described. In some embodiments, the implant can be designed to provide a correction to the patient's existing anatomy in addition to mating with one or more surfaces of the patient anatomy. For example, the treatment planning module 2418 may analyze image data of the patient's native anatomy to determine whether an anatomical correction is needed. The image data may show the patient's native anatomical configuration (e.g., pre-operative anatomy), such as the geometry, orientation, and/or topography of various anatomical features. In some embodiments, for example, the image data may show (and/or be used to determine) various anatomical characteristics, including, but not limited to, vertebral spacing, vertebral orientation, vertebral translation, abnormal bony growth, abnormal joint growth, joint inflammation, joint degeneration, tissue degeneration, stenosis, scar tissue, lumbar lordosis, Cobb angle(s), pelvic incidence, disc height, segment flexibility, rotational displacement, and/or other spinal tissue characteristics. If an anatomical correction is not required, the treatment planning module 2418 can design the implant to fit the patient's native anatomy. If an anatomical correction is required, the treatment planning module 2418 can design the implant such that, when the implant is implanted in the patient, it provides the anatomical correction. Additional details for designing patient-specific implants to provide one or more desired anatomical corrections can be found in U.S. application Ser. No. 16/987,113, filed Aug. 6, 2020, the disclosure of which is incorporated by reference herein in its entirety.

In some embodiments, the generated implant design is a design for an entire device. Alternatively, the generated design can be for one or more components of a device (e.g., coupler, a plate, or a cage), rather than the entire device. In some embodiments, the implant design is for one or more patient-specific device components that can be used with standard, off-the-shelf components. In some embodiments, the generated design is for a patient-specific medical device that can be used with a standard, off-the-shelf delivery instrument. For example, the implants (e.g., screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The implants described herein are expected to improve delivery into the patient's body, placement at the treatment site, and/or interaction with the patient's anatomy.

The computing device 2402 and/or the server 2406 can also design a patient-specific surgical plan (“surgical plan”) based on the patient data set 2408, the surgical team data set 2410, and/or the facility data set 2412. The surgical plan can include a detailed procedure for implanting the implant to a specific target position within the patient. For example, the surgical plan can include aspects of a pre-operative plan (e.g., detection and measurement of patient's anatomy, preparation of patient for a surgical procedure, etc.), a surgical procedure, a surgical approach (e.g., implant technique), one or more surgical steps (preparing tissue for an incision, making an incision, making a resection, removing tissue, manipulating tissue, performing a corrective maneuver, delivering the implant to a target site, deploying the implant at the target site, adjusting the implant at the target site, manipulating the implant once it is implanted, securing the implant at the target site, explanting the implant, suturing tissue, etc.) a target position, site, or location of the implant (e.g., a location, orientation, etc.), and/or other aspects related to pre-operative, operative, or post-operative plans.

In some embodiments, the surgical plan includes an orthopedic surgical procedure such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as PLIF, ALIF, TLIF, LLIF, DLIF, and/or XLIF. Spinal surgery can also include non-fusion surgeries, such as artificial disc replacements. In some embodiments, the surgical procedure includes descriptions of and/or instructions for performing one or more aspects of a patient-specific surgical procedure. For example, the surgical procedure can include one or more of a surgical approach, a corrective maneuver, or a bony resection.

In some embodiments, the surgical plan includes a target position of the implant, delivery paths, intra-vivo implant articulation/movement, unlocking/locking positions, etc. In some embodiments, the surgical plan optionally includes a recommendation to remove tissue to clear space for the implant at the target position. For example, the surgical plan may include instructions to perform an osteotomy, muscular resection, soft tissue detachment, soft tissue retraction, or the like to prepare the patient to receive the patient-specific implant. In some embodiments, the surgical plan includes a manipulation of tissue to prepare the patient to receive the implant. For example, the surgical plan may include instructions to adjust a relative position of two vertebrae, increase a distance between two vertebrae, or the like.

In some embodiments, the surgical plan includes machine-readable instructions for carrying out various steps of the surgical plan. The machine-readable instructions can be configured such that, when executed by a surgical robotic platform, the machine-readable instructions cause the surgical robotic platform to execute various aspects of an operative procedure associated with implanting the implant. For example, the surgical platform may prepare tissue for an incision, make an incision, make a resection, remove tissue, manipulate tissue, perform a corrective maneuver, deliver the implant to a target site, deploy the implant at the target site; adjust a configuration of the implant at the target site, manipulate the implant once it is implanted, secure the implant at the target site, explant the implant, suture tissue, or the like. The instructions may therefor include particular instructions for articulating robotic arms, instruments, and/or tools to perform or otherwise aid in the delivery of the patient-specific implant.

In some embodiments, the surgical plan includes step-by-step written, verbal, and/or graphic instructions that show a surgeon how to perform the patient-specific surgical plan. The patient-specific surgical plan can be displayed to the surgeon before and/or during the operative procedure (e.g., via display 2422). In some embodiments, the written, verbal, and/or graphic instructions can be encoded in computer-readable instructions. The encoded instructions can be decoded and displayed to the surgeon before and/or during the operative procedure. In some embodiments, the patient-specific surgical plan includes both machine-readable instructions and written, verbal, and/or graphic illustrations.

In some embodiments, the computing system 2400 may consider one or more reference data sets when designing the patient-specific implant and/or the patient-specific surgical plan. For example, in some embodiments the server 2406 includes at least one database 2420 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 2420 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, preferences, and/or any other information or parameters relevant to the reference patient, such as any of the data described herein with respect to the patient data set 2408. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine. As another example, a reference patient data set can include treatment data regarding at least one treatment procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, activity level, return to work, complications, recovery times, efficacy, mortality, and/or follow-up surgeries.

In some embodiments, the server 2406 receives at least some of the reference patient data sets from a plurality of healthcare provider computing systems. Each healthcare provider computing system can include at least one reference patient data set (e.g., reference patient data sets) associated with reference patients treated by the corresponding healthcare provider. The reference patient data sets can include, for example, kinematic records, electronic medical records, electronic health records, biomedical data sets, etc.

In embodiments in which the implant and/or the surgical plan is designed based on the reference data, the data analysis module 2416 can include one or more algorithms for identifying a subset of reference data from the database 2420 that is likely to be useful in developing a treatment plan. For example, the data analysis module 2416 can compare patient-specific data (e.g., the patient data set 2408 received from the computing device 2402) to the reference data from the database 2420 (e.g., the reference patient data sets) to identify similar data (e.g., one or more similar patient data sets in the reference patient data sets). The comparison can be based on one or more parameters, such as age, gender, BMI, pathology, kinematics, lumbar lordosis, pelvic incidence, and/or treatment levels. The parameter(s) can be used to calculate a similarity score for each reference patient. The similarity score can represent a statistical correlation between the patient data set 2408 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients. In some embodiments, the data analysis module 2416 includes one or more algorithms that select a set or subset of the reference patient data based on criteria other than patient parameters, such as the surgical team data set 2410 (e.g., based on surgeon expertise, outcomes of particular types of procedures performed by the surgeon, etc.) and/or the facility data set 2412 (e.g., surgical equipment such as surgical robots).

The data analysis module 2416 can further be configured with one or more algorithms to select a subset of the reference patient data sets, e.g., based on similarity to the patient data set 2408 and/or treatment outcome of the corresponding reference patient. For example, the data analysis module 2416 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, range of motion, kinematic data, HRQL, activity level, complications, recovery times, efficacy, mortality, or follow-up surgeries. As described in further detail below, in some embodiments, the data analysis module 2416 calculates an outcome score by assigning values to each outcome parameter. A patient can be considered to have a favorable outcome if the outcome score is above, below, or at a specified threshold value.

In some embodiments, the data analysis module 2416 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 2416 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and/or other data sets can be utilized. In another example, the patient-specific treatment plan can be generated based on available robotic surgical systems. The reference patient data sets can be selected based on patients that have been operated on using comparable robotic surgical systems under similar conditions (e.g., size and capabilities of surgical teams, hospital resources, etc.).

In embodiments in which the implant and/or the surgical plan is designed based on the reference data, the treatment planning module 2418 can include one or more algorithms that generate the implant and/or the surgical plan based on the reference data. In some embodiments, the treatment planning module 2418 is configured to develop and/or implement at least one predictive model for generating the treatment plan, also known as a “prescriptive model.” The predictive model(s) can be developed using clinical knowledge, statistics, machine learning, AI, neural networks, or the like. In some embodiments, the output from the data analysis module 2416 is analyzed (e.g., using statistics, machine learning, neural networks, AI, etc.) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a treatment plan will produce a favorable outcome for the particular patient. The predictive model(s) can be validated, e.g., by inputting data into the model(s) and comparing the output of the model to the expected output.

In some embodiments, the treatment planning module 2418 is configured to generate the implant design based on previous treatment data from reference patients. For example, the treatment planning module 2418 can receive a selected subset of reference patient data sets and/or similar patient data sets from the data analysis module 2416 and determine or identify treatment data from the selected subset. The treatment data can include, for example, range of motion and/or other kinematic data, treatment procedure data (e.g., surgical procedure or intervention data) and/or medical device design data (e.g. implant design data) that are associated with favorable or desired treatment outcomes for the corresponding patient. The treatment planning module 2418 can analyze the treatment procedure data and/or medical device design data to determine an optimal treatment protocol for the patient to be treated. For example, the treatment procedures and/or medical device designs can be assigned values and aggregated to produce a treatment score. The patient-specific treatment plan can be determined by selecting treatment plan(s) based on the score (e.g., higher or highest score; lower or lowest score; score that is above, below, or at a specified threshold value). The personalized treatment plan can be based on, at least in part, the patient-specific technologies or patient-specific selected technology.

Alternatively or in combination, the treatment planning module 2418 can generate the implant designs based on correlations between data sets. For example, the treatment planning module 2418 can correlate implant designs (including articulation designs) and medical device design data from implant designs for similar patients with favorable outcomes (e.g., as identified by the data analysis module 2416). Correlation analysis can include transforming correlation coefficient values to values or scores. The values/scores can be aggregated, filtered, or otherwise analyzed to determine one or more statistical significances. These correlations can be used to determine treatment procedure(s) and/or medical device design(s) that are optimal or likely to produce a favorable outcome for the patient to be treated.

Alternatively or in combination, the treatment planning module 2418 can generate designs using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 2418 generates the treatment plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 820, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training dataset can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate the treatment plan, the patient data set 2408, the surgical team data set 2410, and/or the facility data set 2412 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one treatment plan for the patient. In embodiments where multiple trained machine learning models are used, the models can be run sequentially or concurrently to compare outcomes and can be periodically updated using training data sets. The treatment planning module 2418 can use one or more of the machine learning models based the model's predicted accuracy score.

To facilitate visualization, the display 2422 can display one or more patient images, virtual models, delivery paths (e.g., overlaid on patient images, in virtual models, etc.), and associated metrics/parameters, including pre-operative metrics/parameters, planned metrics/parameters, and/or post-operative metrics/parameters. As another example, the display 2422 can show a design for a medical device to be implanted in the patient, such as a 2D or 3D model of the device design. The device design can include, for example, a size and configuration of an implant (e.g., footprint of intervertebral cage), length and curvature of spinal rods, length and characteristics of screws (e.g., bone screws, pedicle screws), range of motion of artificial discs, or the like. The display 2422 can also show patient information, such as 2D or 3D images or models of the patient's anatomy where the surgical procedure is to be performed and/or where the device is to be implanted. In some procedures, the system can design implants based on received third-party data related to the size of the implant. For example, the size of the implant (e.g., an interbody cage or an artificial disc) can be a percentage of vertebral endplate(s), percentage of original disc height prior to surgery, or the like. In some procedures, the system can design implants based on received third-party data related to the curvature of the patient's spine. For example, a length, curvature, and strength of the implants (e.g., spinal rods) can be designed based on the received third-party curvature data. Coupling features can be designed after designing a main body of the implant.

The display 2422 can display recommended delivery paths, types of implants, procedures (e.g., levels of treatment in spine procedures), placement criteria, contact interface criteria, loading criteria, properties of implants, characteristics of implants, or the like. In spine procedures, the type of implant can be, for example, a fixed intervertebral cage, an artificial disc, a rod, etc. selected based on whether the system determines there should be mobility at spinal levels. The placement criteria can include, for example, distance from anatomical features, position with respect to the perimeter of a vertebral endplate or body, and/or other positional location. In some spinal fusion procedures, the coverage criteria can include, for example, maximum or minimum percentage of coverage of the vertebral endplate, areas of coverage along the vertebral endplate, or the like. The load-bearing characteristics can include, for example, strength of the implant, fracture toughness of the implant, fatigue life of the implant, degrees of motion of the implant, or the like. The characteristics of the implant can include, for example, lattice characteristics (e.g., density of lattice structure, distribution of lattice structure), openings for receiving grafting material, or the like. The display 2422 can display recommendations for other procedures.

The platform can perform one or more simulations using 3D models, labeled patient images, modified patient images, or the like. The platform 2409 can measure the patient's anatomy based on the 3D models or modified patient images (e.g., patient images with anatomical features repositioned). In some embodiments, the platform 2409 can build virtual implants using the third-party data or a combination of the third-party data and the patient's data. The virtual implants and 3D models of the patient can be used to generate one or more simulations. The simulations can be dynamic/biomechanical simulations of the patient with real-time data (e.g., weight, BMI, etc.) collected from a user device (e.g., wearable device, Internet of Things (IoT) device, etc.) to predict the success of installing an implant in the patient. The platform 2409 can evaluate the dynamic/biomechanical simulations to determine, for example, whether additional third-party data should be collected, whether additional relevant third-party data is available, etc. In response to determining that additional data should be collected or is available, the platform 2409 can search for data, determine whether to terminate the search (e.g., to limit usage of processing resources), determine whether to generate target searches to manage resources, and/or delete all or some of the collected data to manage memory usage. The platform 2409 can collect data (e.g., periodically or continuously collect data) for one or more time periods, a pre-manufacturing period, a pre-operative time period, a post-operative time period, etc.

The platform 2409 can determine whether to use available information. The determination can be based on one or more of a confidence score for the available data (e.g., a confidence score exceeding a threshold score), user setting value (e.g., a user-inputted search alert query), etc. The system can notify the user of the newly available data. In response to the system determining that the newly available third-party data should be used, the user can be notified prior to generating a newly modified treatment plan based on the newly available data. The system can modify treatments in real-time or near real-time so that the user can determine in real-time or near real-time whether to pursue the planned treatment or a newly modified treatment. In another example, the user can be notified that newly available third-party data could be used to modify designs of one or more implants or instruments. The treatment planning module 2418 can update, modify, or replace the implant design and/or the surgical plan based on the newly available third-party data.

The implant design and/or the surgical plan generated by the treatment planning module 2418 can be transmitted via the communication network 2404 to the computing device 2402 for output to a user (e.g., clinician, surgeon, healthcare provider, patient). In some embodiments, the computing device 2402 includes or is operably coupled to a display 2422. The display 2422 can show various aspects of a surgical procedure to be performed on the patient, such as the surgical approach, treatment levels, corrective maneuvers, tissue resection, and/or implant placement. To facilitate visualization, a virtual model of the surgical procedure can be displayed. Additionally or alternatively, the display 2422 can show a design for the implant 2461, such as a two- or three-dimensional model of the device design. The display 2422 can also show patient information, such as two- or three-dimensional images or models 2429 of the patient's anatomy where the surgical procedure is to be performed and/or implant locations 2432 where the device is to be implanted. The model 2429 can be a virtual model (e.g., a 2D virtual model, a 3D virtual model, etc.) representing the patient's anatomy. The virtual model can be CAD model generated using CAD software, patient images, etc. The display 2422 can also display structural features of the implant suitable for contacting anatomical features to improve treatment, reduce implant movement, etc. The structural features can be rigid surfaces (e.g., outer surfaces of an implant body), anchors, fixation features, etc. Images of the implantation site can be analyzed to identify such anatomical features identified by the treatment planning module 2418. The computing device 2402 can further include one or more user input devices (not shown) allowing the user to modify, select, approve, and/or reject the displayed treatment plan(s).

The display 2422 can also show surgical plans 2457, patient metrics 2423 (e.g., pre-operative metric, predicted post-operative metrics, etc.), feedback buttons 2465 (e.g., approve button, rejection, modify button, etc.), implant design 2461, and/or patient data 2427. The surgical plans 2457 displayed can include the number of instruments, configuration of instruments, surgical paths, in-vivo implant articulation, implant locking/unlocking positions, and/or surgical techniques used based on the condition being treated. The surgical paths displayed can include, for example, lateral approaches, transforaminal approaches, and anterior approaches that can be used to access and treat the cervical spine, thoracic spine, etc. The surgical paths displayed can also include positional information (e.g., side-to-side motion, rotation, motion associated with pushing distally, motion associated with pulling proximally, etc.). Positional information can include, for example, positional information of the implant, positional information of the instrument, positional information of the delivery tools, positional information of anatomic elements, or the like. The implant design 2461 can include the number of implants and/or the configurations of implants. The configuration of implants can include the shape, dimensions (e.g., length, curvature, etc.), material properties, etc.

The display 2422 can also show patient information, such as two- or three-dimensional images or models 2429 of the patient's anatomy where the surgical procedure is to be performed and/or implant locations 2432 where the device is to be implanted. The display 2422 can also display structural features of the implant suitable for contacting anatomical features to improve treatment, reduce implant movement, etc. Images of the implantation site can be analyzed to identify such anatomical features identified by the treatment planning module 2418.

The surgical plan 2457 can also include surgical information, surgical steps (e.g., surgical implant procedure, target implant location and/or orientation, etc.), technology recommendations (e.g., device and/or instrument recommendations), and/or medical device designs. For example, the surgical plan can include at least one candidate treatment procedure (e.g., a surgical procedure or intervention) and/or at least one medical device (e.g., an implanted medical device (also referred to herein as an “implant” or “implanted device”) or implant delivery instrument). Lateral approaches, transforaminal approaches, and anterior approaches can be used to access the cervical spine, thoracic spine, etc. The number of instruments, configurations of instruments, implants, and/or surgical techniques can be selected based on the condition to be treated. The design for the implant 2461 can be a two- or three-dimensional model of the device design.

In some embodiments, the patient-specific implant design generated by the treatment planning module 2418 can be transmitted from the computing device 2402 and/or the server 2406 to a manufacturing system 2424 for manufacturing a corresponding medical device. The manufacturing system 2424 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. For example, the manufacturing system 2424 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 2424 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 2424 can manufacture one or more patient-specific medical devices based on fabrication instructions or data (e.g., CAD data, 3D data, digital blueprints, stereolithography data, or other data suitable for the various manufacturing technologies described herein). In some embodiments, the patient-specific medical device can include features, materials, and/or designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components (e.g., pro-fabricated couplers) and the selected pre-fabricated components can be modified based on the fabrication instructions or data.

The treatment plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 818 generates control instructions configured to cause a surgical robot (e.g., robotic surgery systems, navigation systems, etc.) to partially or fully perform a surgical procedure. The control instructions can be transmitted to the robotic apparatus by the computing device 2402 and/or the server 2406.

Following the treatment of the patient in accordance with the treatment plan, treatment progress can be monitored over one or more time periods to update the data analysis module 2416 and/or treatment planning module 2418. Post-treatment data can be added to the reference data stored in the database 2420. The post-treatment data can be used to train machine learning models for developing patient-specific treatment plans, patient-specific medical devices, or combinations thereof.

It shall be appreciated that the components of the computing system 2400 can be configured in many different ways. For example, in alternative embodiments, the database 2420, the data analysis module 2416 and/or the treatment planning module 2418 can be components of the computing device 2402, rather than the server 2406. As another example, the database 2420, the data analysis module 2416, and/or the treatment planning module 2418 can be located across a plurality of different servers, computing systems, or other types of cloud-computing resources, rather than at a single server 2406 or computing device 2402.

Additionally, in some embodiments, the computing system 2400 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like. In some embodiments, the computing system 2400 may include additional features and/or capabilities, such as any of those described in U.S. application Ser. No. 16/735,222, filed Jan. 6, 2020, the disclosure of which is incorporated by reference herein in its entirety.

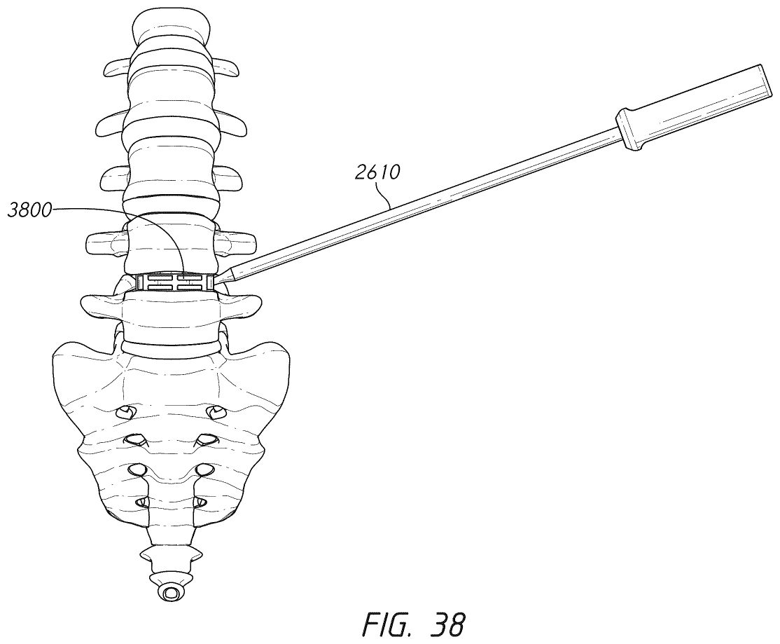

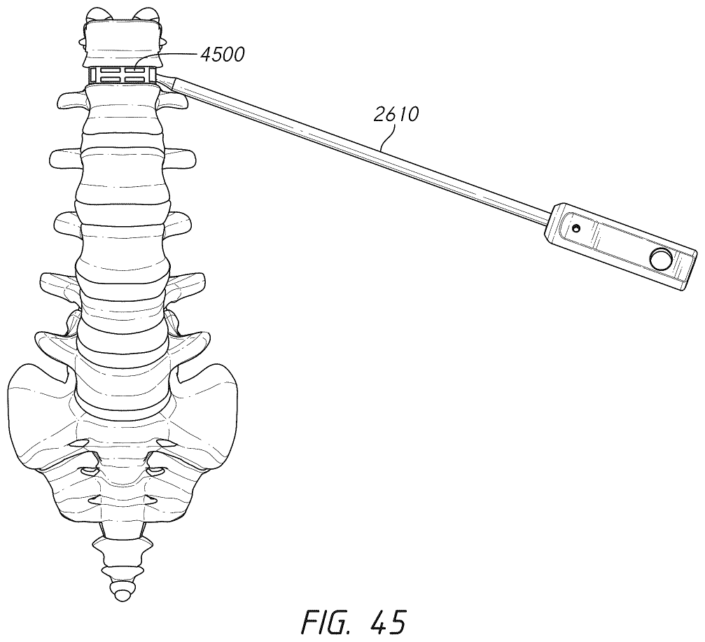

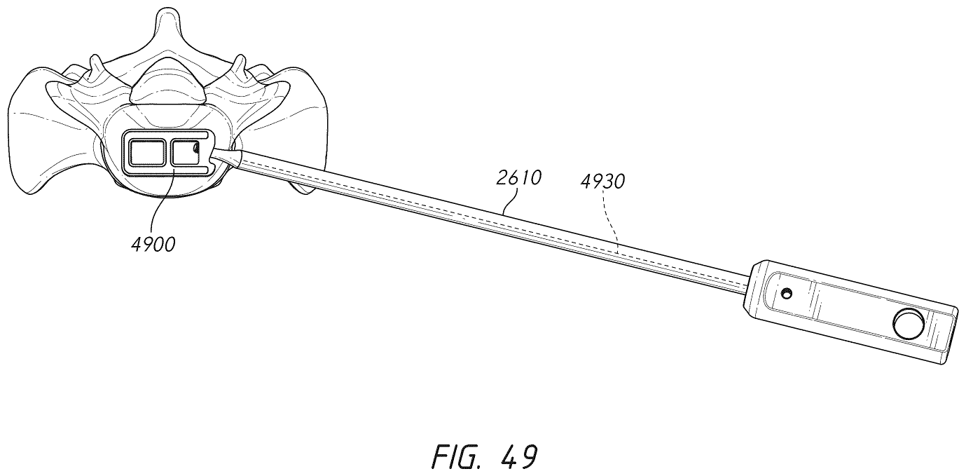

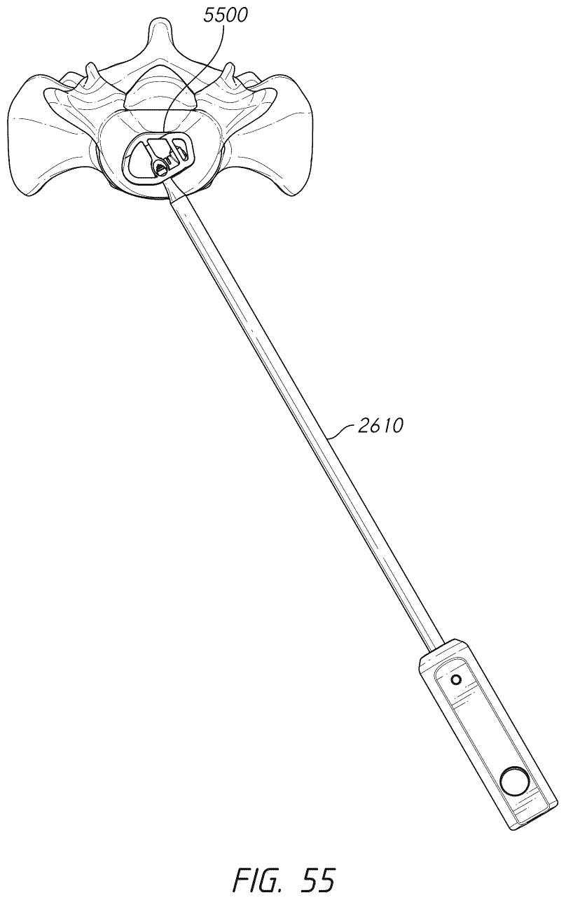

The computing system 2400 can also design items based on surgical approaches to assist with delivering implants to target implantation sites, generating surgical plans, and/or displaying surgical techniques/instructions. The computing system 2400 can generate a surgical approach-specific implant design (or multiple surgical approach-specific implant designs) based on the target anatomical correction and planned surgical approach (e.g., ALIF approach, LLIF approach, OLIF approach, ALIF approach, etc.). The implants can have angled coupling features for achieving angular positioning of the implants relative to, for example, a longitudinal axis of an insertion or inserter instrument, delivery instrument, etc. The implants can be angled to perform, for example, high-pelvic incidence ALIF procedures (FIGS. 25-37), high iliac crest LLIF procedures (FIG. 38-44), low rib LLIF procedures (FIGS. 45-48), OLIF procedures (FIGS. 49-54), lateral ALIF/OLIF procedures (FIGS. 55-64), and other procedures disclosed herein. In some procedures, the same surgical approach can be used to deliver implants to different levels along the spine. In some procedures, different surgical approaches can be used to deliver implants to different levels along the spine.

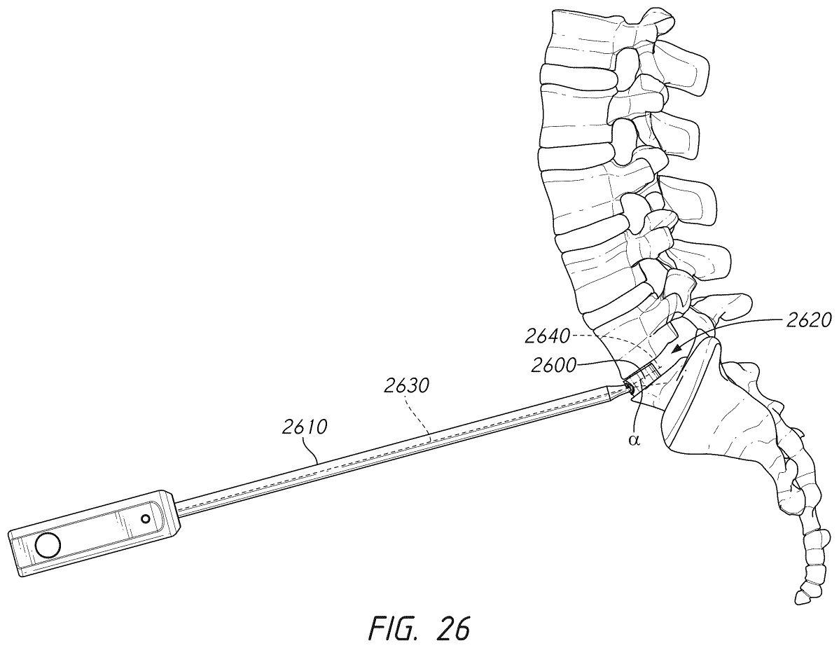

FIG. 25 is a side view of anatomy and an implant 2600 with a transverse plane 2640 designed for an ALIF approach in accordance with at least some embodiments of the technology. FIG. 26 shows the implant 2600 at a target implantation site along a spine. Referring to FIGS. 25 and 26, the implant 2600 is detachably coupled to a delivery or insertion instrument 2610 such that the implant 2600 is held at an angular orientation matching a disc space 2620. In some embodiments, the implant 2600 can be detachably coupled to the insertion instrument 2610 at an angular orientation with respect to a longitudinal axis 2630 (FIGS. 26 and 31) of the insertion instrument 2610. For example, the transverse plane 2640 of the implant can be positioned at an angle α relative to the longitudinal axis 2630 of the insertion instrument 2610. This configuration aligns the implant 2600 along a delivery path suitable for the ALIF approach towards and/or into the disc space 2620. The implant 2600 can be moved along a planned ALIF delivery path 2630 and inserted into the disc space 2620. The transverse plane 2640 or a longitudinal axis of the implant 2600 can be generally parallel to the disc space 2620 and/or adjacent vertebral endplate(s). After positioning the implant 2600 at the target implantation site, the insertion instrument 2610 can be rotated relative to the implant 2600 to detach the insertion instrument 2610 from the implant 2600.

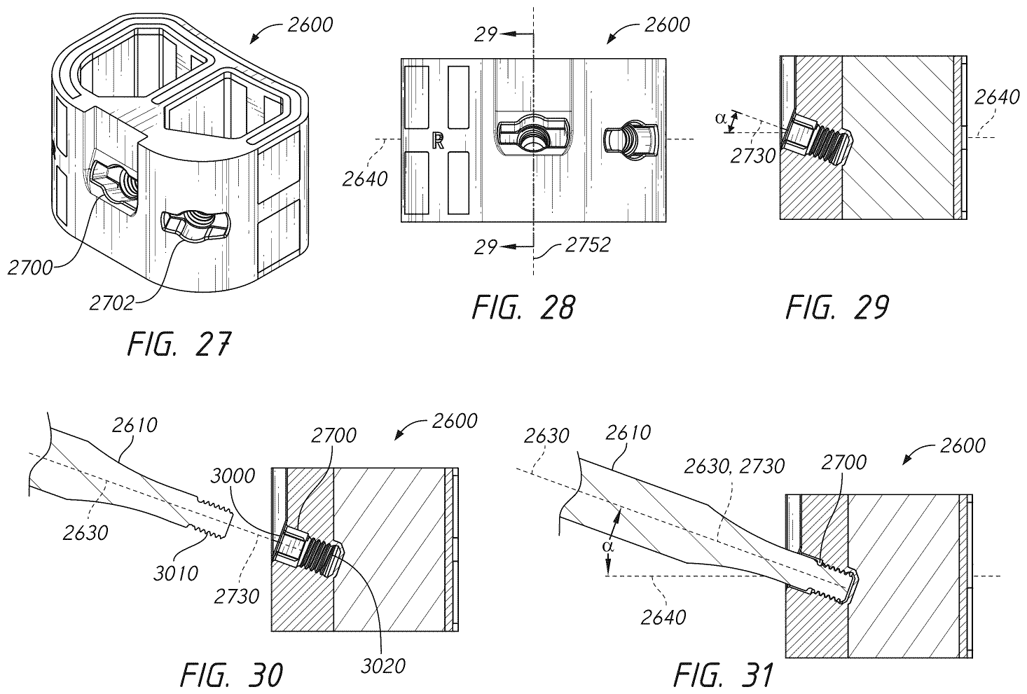

FIG. 27 is an isometric view of the implant 2600 in accordance with at least some embodiments of the technology. FIG. 28 is a front view of the implant 2600. FIG. 29 is a cross-sectional view of the implant 2600 taken along line 29-29 of FIG. 28. Referring now to FIG. 27, the implant 2600 includes multiple coupling features or elements 2700, 2702. The description of one of the coupling elements 2700, 2702 applies to the other coupling feature unless indicated otherwise. The coupling element 2700 can be positioned along a midplane 2752 (FIG. 28) of the implant 2600 and angled with respect to the transverse plane 2640 (FIGS. 28 and 29) of the implant 2600. Referring to FIG. 29, an angle α can be defined between a longitudinal axis 2730 of the coupling element 2700 (or longitudinal axis 2630 of the insertion instrument 2610 of FIG. 31) and the transverse plane 2640. The longitudinal axis 2730 can be an axis of a passage/hole of the coupling element 2700. The angle α can be selected based on the subject's anatomy and can be, for example, 5 degrees, 10 degrees, 20 degrees, 30 degrees, 40 degrees, 50 degrees, or within ranges encompassing such angles (e.g., a range of 10 degrees to 30 degrees, 10 degrees to 40 degrees, etc.). In some embodiments, the angle α can be selected based on an angle of pelvic incidence measured by a user, the computing system 2400 discussed in connection with FIG. 24, or the like.

FIGS. 30 and 31 are cross-sectional views showing the insertion instrument 2610 and the implant 2600. Referring now to FIG. 30, the insertion instrument 2610 can be inserted into an opening 3000 of the coupling element 2700. The insertion instrument 2610 can include an externally threaded distal tip 3010 configured to threadably engage internal threads 3020 of the coupling element 2700. The insertion instrument 2610 can be rotated in a first direction (e.g., clockwise direction) to advance the distal tip 3010 along the internal threads 3020. As shown in FIG. 31, the insertion instrument 2610 is locked to the implant 2600. As shown in FIGS. 29-31, the longitudinal axis 2630 of the insertion instrument 2610 generally aligns with (e.g., is parallel with) the longitudinal axis 2730 of the coupling element 2700 and angles (e.g., angle α) with respect to the transverse plane 2640 of the implant 2600. Thus, the coupling element 2700 enables the implant 2600 to detachably couple to the insertion instrument 2610 at an angle α relative to the longitudinal axis 2630 of the insertion instrument 2610. The insertion instrument 2610 can be rotated in an opposite second direction (e.g., counterclockwise direction) to decouple the distal tip 3010 from the coupling element 2700.

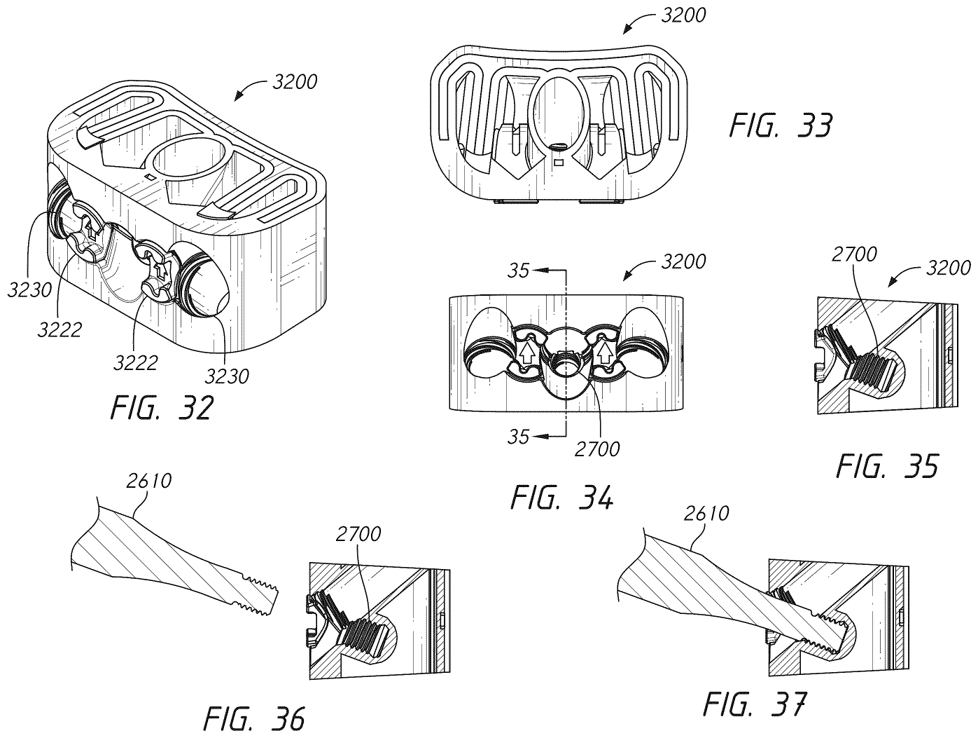

FIG. 32 is an isometric view of an implant 3200 in accordance with at least some embodiments of the present technology. FIG. 33 is a top view of the implant 3200. FIG. 34 is a front view of the implant 3200. FIG. 35 is a cross-sectional view of the implant 3200 taken along line 35-35 of FIG. 34. Referring now to FIGS. 32-35, the implant 3200 includes an angled coupling element 2700 and one or more locking cams 3222 configured to hold screws (e.g., anchor screws, bone screws) in screw receiving features 3230 (e.g., openings, threaded through-holes, etc.). PCT App. No. PCT/US2024/010202, filed Jan. 3, 2024, discloses implants, intervertebral spacers, anchors, screws, locking cams, features, and methods that can be used with, or incorporated into, the present technology. PCT App. No. PCT/US2024/010202 is incorporated by reference in its entirety.

FIGS. 36 and 37 are cross-sectional views showing the insertion instrument 2610 and the implant 3200. The locking cams 3222 allow insertion of the insertion instrument 2610 into the coupling feature, as discussed in connection with FIGS. and 30-31. After separating the insertion instrument 2610 and the implant 3200, the bone screws can be installed and held in place using the locking cams 3222.

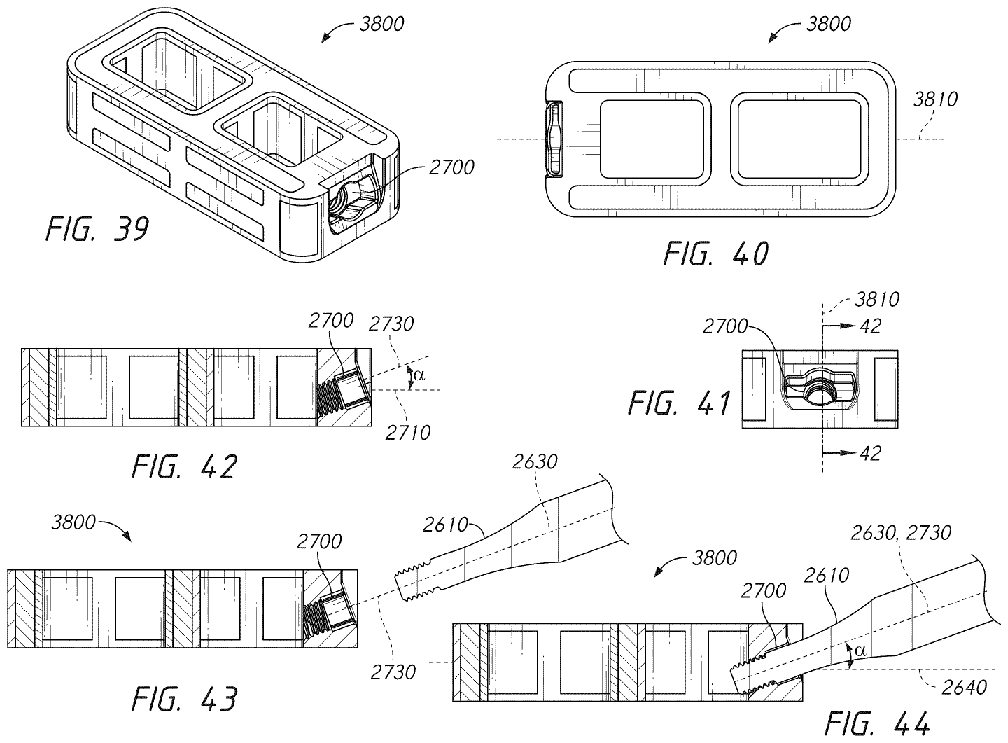

FIG. 38 is a front view of anatomy and an implant 3800 designed for a high iliac crest LLIF procedure in accordance with at least some embodiments of the technology. FIG. 39 is an isometric view of the implant 3800 in accordance with at least some embodiments of the present technology. FIG. 40 is a bottom view of the implant 3800 of FIG. 39. FIG. 41 is a front view of the implant 3800 of FIG. 39. FIG. 42 is a cross-sectional view of the implant 3800 taken along line 42-42 of FIG. 41. FIGS. 43 and 44 are cross-sectional views showing the insertion instrument 2610 coupling to the implant 3800. Referring now to FIGS. 41-44, the implant 3800 includes a coupling element 2700 positioned along a sagittal plane 3810 of the implant 3800. The coupling element 2700 can be angled upwardly with respect to the transverse plane 2640 (FIG. 42) of the implant 3800. For example, an angle α (FIG. 42) defines the relationship between the longitudinal axis 2730 of the coupling element 2700 and the transverse plane 2640. This angle α also determines the orientation of the coupling element 2700 relative to the longitudinal axis 2730 along a delivery path for a surgical approach and/or relative to the longitudinal axis 2630 of the insertion instrument 2610 (FIG. 44). In some embodiments, the longitudinal axis 2630 (FIGS. 43 and 44) of the insertion instrument 2610 is generally parallel to the longitudinal axis 2730 of the coupling element 2700. The angle α can be selected based on the subject's anatomy and can be, for example, in a range of 10 degrees to 30 degrees, 15 degrees to 35 degrees, or other ranges when the connecting line of the bilateral iliac crest is higher than, for example, the horizontal midline of the L5 pedicle.

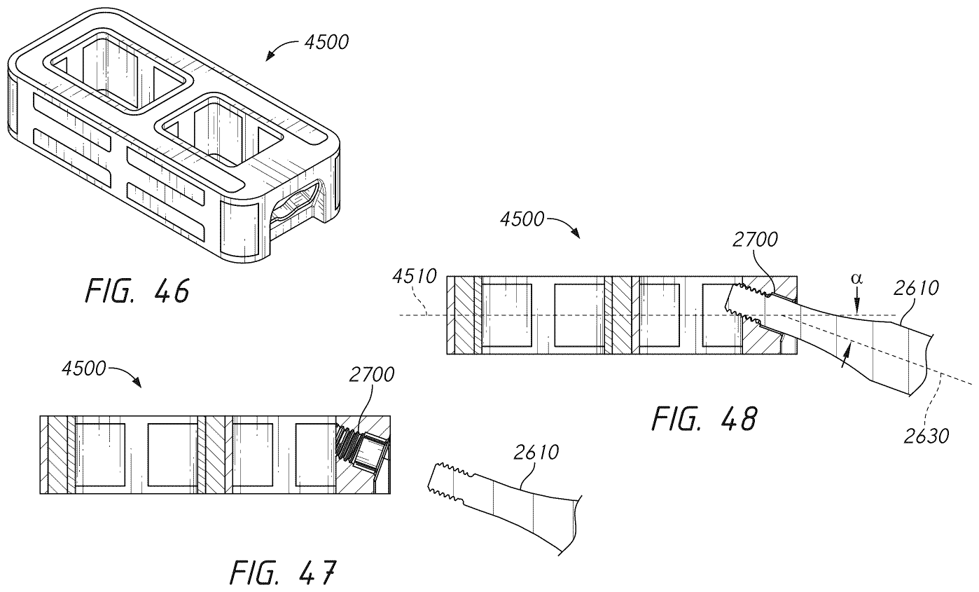

FIG. 45 is a front view of anatomy and an implant 4500 designed for a low rib LLIF procedure in accordance with at least some embodiments of the technology. FIG. 46 is an isometric view of the implant 4500 in accordance with at least some embodiments of the present technology. FIGS. 47 and 48 are cross-sectional views of the implant 4500 showing the insertion instrument 2610 coupling to the implant 4500. As shown in FIG. 48, the coupling element 2700 is angled downwardly at an angle α away from a transverse plane 4510. The angle α can be selected based on the subject's anatomy and the desired orientation of the implant 4500 relative to the longitudinal axis 2630 of the insertion instrument 2610. The angle α can be in a range of 10 degrees to 30 degrees, 15 degrees to 35 degrees, or other ranges of angles.

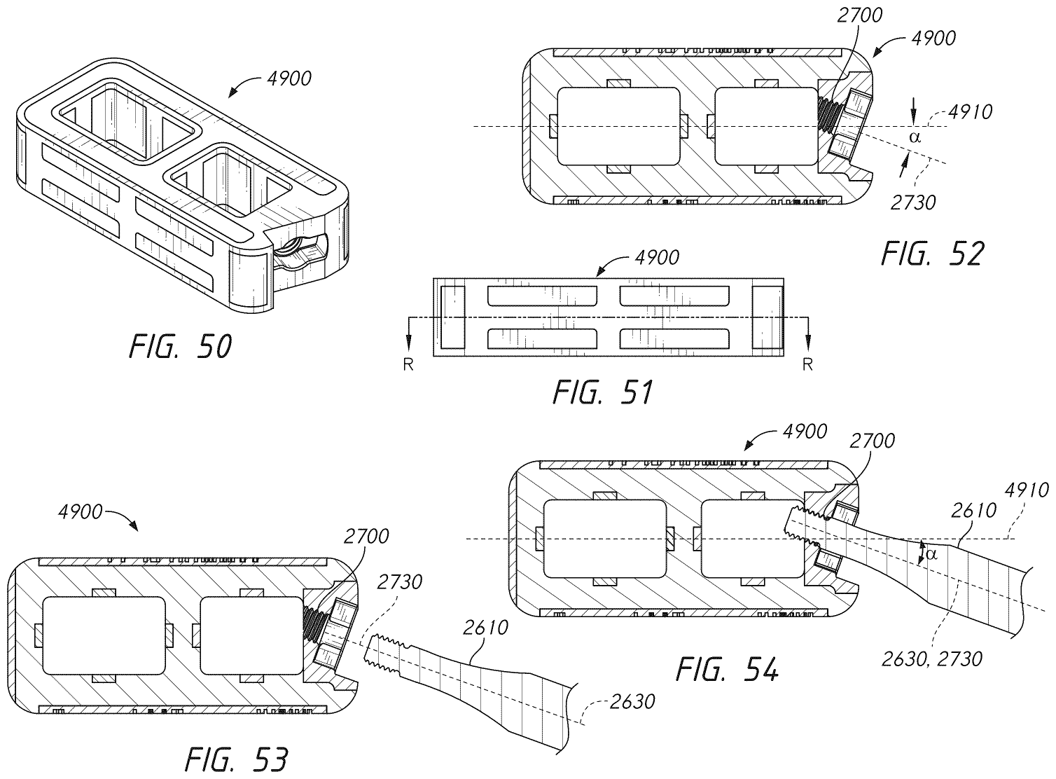

FIG. 49 is a top view of anatomy and an implant 4900 designed for an OLIF procedure in accordance with at least some embodiments of the technology. FIG. 50 is an isometric view of the implant 4900 in accordance with at least some embodiments of the present technology. FIG. 51 is a side view of the implant 4900. FIG. 52 is a cross-sectional view of the implant 4900 taken along line 52-52 of FIG. 51. FIGS. 53 and 54 are cross-sectional views showing the insertion instrument 2610 coupling to the implant 4900. Referring now to FIGS. 52-54, the implant 4900 includes a coupling element 2700 positioned at a lateral angle α with respect to the plane 4910 (midplane or sagittal plane) of the implant 4900 and/or a lateral angle α relative to the longitudinal axis 2730 of the coupling element 2700. As described herein, the longitudinal axis 2730 of the coupling element 2700 is generally parallel to the longitudinal axis 2630 of the insertion instrument 2610, thereby orienting the implant 4900 relative to the longitudinal axis 2630 of the insertion instrument 2610. The angle α can be selected based on the subject's anatomy and can be, for example, in a range of 10 degrees to 30 degrees, 15 degrees to 35 degrees, or other ranges of angles such that the insertion instrument 2610 is positionable along an ALIF/OLIF delivery path 4930 (FIG. 49).

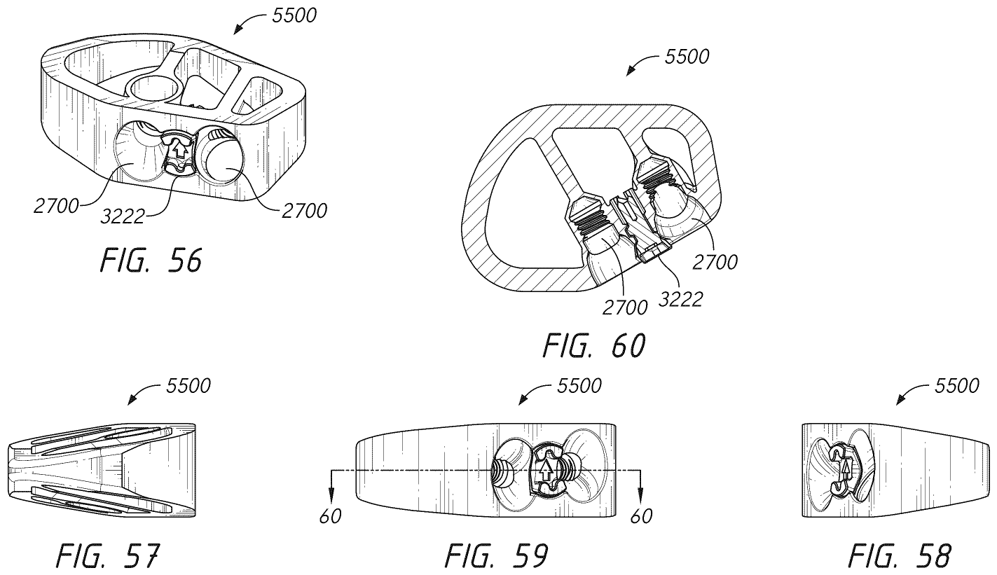

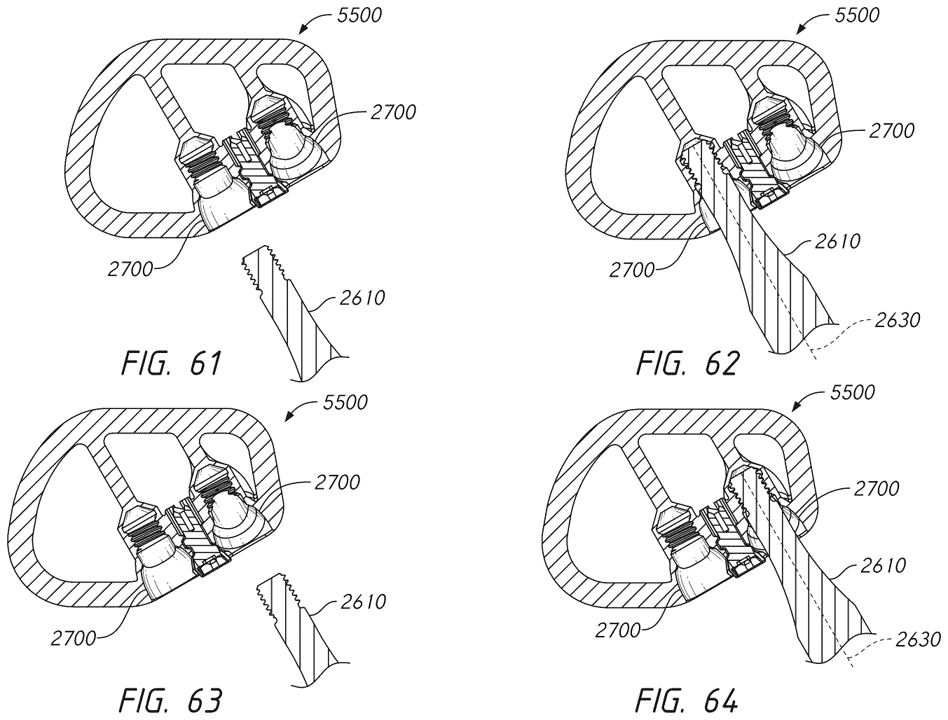

FIG. 55 is a top view of anatomy and an implant 5500 designed for a lateral procedure (e.g., lateral ALIF/OLIF procedure) in accordance with at least some embodiments of the technology. FIG. 56 is an isometric view of the implant 5500 in accordance with at least some embodiments of the present technology. FIGS. 57 and 58 are opposite side views of the implant 5500 of FIG. 56. FIG. 59 is a front view of the implant 5500 of FIG. 56. FIG. 60 is a cross-sectional view of the implant 5500 taken along line 60-60 of FIG. 59. FIGS. 61-64 are cross-sectional views showing the insertion instrument 2610 coupling to different coupling elements 2700 of the implant 5500. Referring to FIGS. 56-58, the implant includes multiple coupling elements 2700. The coupling elements 2700 can also be configured to receive anchors, such as bone screws. The implant 5500 can include a locking cam 3222 configured to retain the bone screws. FIGS. 61 and 62 show the insertion instrument 2610 coupling to the left coupling element 2700. FIGS. 63 and 64 show the insertion instrument 2610 coupling to the right coupling element 2700. One or both of the left and right coupling elements 2700 can detachably couple to the implant 5500, positioning it at an angular orientation relative to the longitudinal axis 2630 of the insertion instrument 2610 (FIGS. 62 and 64), and as described in more detail above. The number, positions, and configurations of coupling features can be selected based on the procedure to performed, desired surgical flexibility/options, maneuverability of the implant, available surgical robots, navigation equipment, or the like.

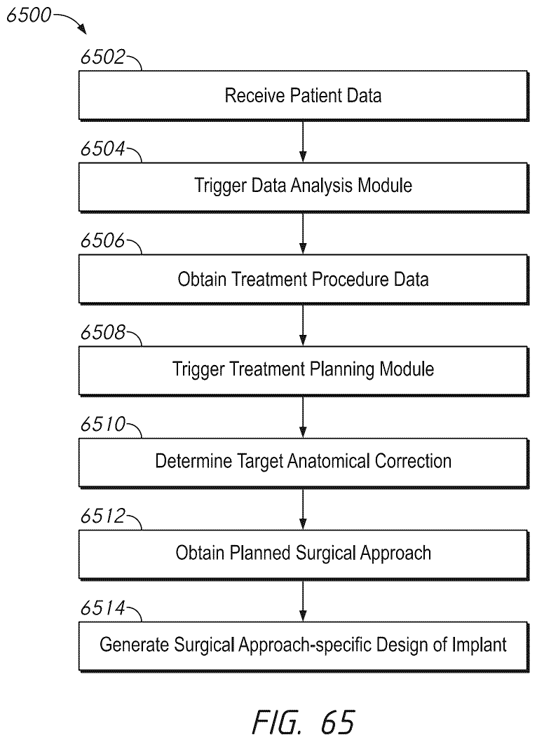

FIG. 65 is a flow diagram illustrating an example process 6500 for designing a surgical approach-specific implant in accordance with at least some embodiments of the technology. In some embodiments, the process 6500 is performed by a computing system, for example, the computing system 2400 of FIG. 24. Additionally or alternatively, one or more of the steps described herein can be initiated and/or executed by a user, a computing device (e.g., the computing device 2402 of FIG. 24), or any other computing device in a network of the computing system (e.g., the communication network 2404 of FIG. 24). As described below, the process 6500 is initiated and executed by a computing system with user interaction at one or more steps of the process 6500. However, one skilled in the art will appreciate that, for this process and other processes and methods disclosed herein, the functions performed in the processes and methods may be performed entirely by the computing system, the computing device, another computing device, and/or in a differing order. Furthermore, the outlined steps and operations are provided only as examples, and some of the steps and operations may be optional, combined into fewer steps and operations, or expanded into additional steps and operations without detracting from the essence of the disclosed embodiments.

Initially, at step 6502, a computing system receives patient data. For example, patient data can include one or more images, medical history, and similar information stored in a patient database of the computing system and/or associated with the patient's ID. In some embodiments, the patient data is stored in a database accessible by the computing system (e.g., database 2420 of FIG. 24) within the network the computing system is part of.

At step 6504, the computing system triggers a data analysis module. For example, the data analysis module can be the data analysis module 2416 of FIG. 24. The data analysis module can access the database described above and herein to acquire additional patient data, treatment procedure data, and/or the like, in addition to the patient data received.

At step 6506, the computing system obtains treatment procedure data from the database based on the received patient data. For example, the treatment procedure data can be surgical procedure data, intervention data, and/or medical device design data (e.g., implant design data) that are associated with favorable or desired treatment outcomes for the patient.

At step 6508, the computing system triggers a treatment planning module. For example, the treatment planning module can be the treatment planning module 2418 of FIG. 24. The treatment planning module can receive data from the data analysis module, including the patient data and treatment procedure data acquired.

At step 6510, the computing system determines a target anatomical correction based on the patient data received, the treatment procedure data, and/or the like. For example, the treatment planning module can analyze image data of the patient's native anatomy to determine whether an anatomical correction is needed. The image data can show the patient's native anatomical configuration (e.g., pre-operative anatomy), such as the geometry, orientation, and topography of various anatomical features. In some embodiments, the image data can show and/or can be used to determine various anatomical characteristics, including, but not limited to, vertebral spacing, vertebral orientation, vertebral translation, abnormal bony growth, abnormal joint growth, joint inflammation, joint degeneration, tissue degeneration, stenosis, scar tissue, lumbar lordosis, Cobb angle(s), pelvic incidence, disc height, segment flexibility, rotational displacement, and other spinal tissue characteristics. The treatment planning module can analyze the image data received using the treatment procedure data acquired. For example, the treatment procedure data can include historical image and procedure data corresponding to the anatomical features being analyzed. The computing system can compare this historical data to the patient's anatomy to determine if and/or where anatomical correction is necessary. The computing system can further use the data acquired to determine and/or select the target anatomical correction. Additionally or alternatively, the computing system can determine a target location for an implant based on the target anatomical correction, physician input, or the like.

At step 6512, the computing system obtains a planned surgical approach. In some embodiments, the planned surgical approach can be selected by a user or an operator of the computing system, for example, a physician or healthcare professional performing and/or assisting in an intervertebral procedure. The user can manually select a surgical approach via a user device. In some embodiments, the treatment planning module automatically determines one or more viable surgical approaches based on the target anatomical correction and displays the surgical approaches on the user device for the user to select from. Additionally or alternatively, the computing system can automatically obtain the planned surgical approach based on, for example, a machine learning module that determines the surgical approach with the highest outcome score (i.e., highest likelihood for achieving anatomical correction). The surgical approaches can be used to achieve the target anatomical correction and can include delivering one or more implants using ALIF, LLIF, XLIF, TLIF, and/or PLIF approaches, as described in more detail with reference to FIGS. 25-64. In some embodiments, the approaches are associated with one or more historical surgical procedures that can be used for planning surgical approach-specific implants, implantation instruments, delivery paths, surgical plans, and/or the like.

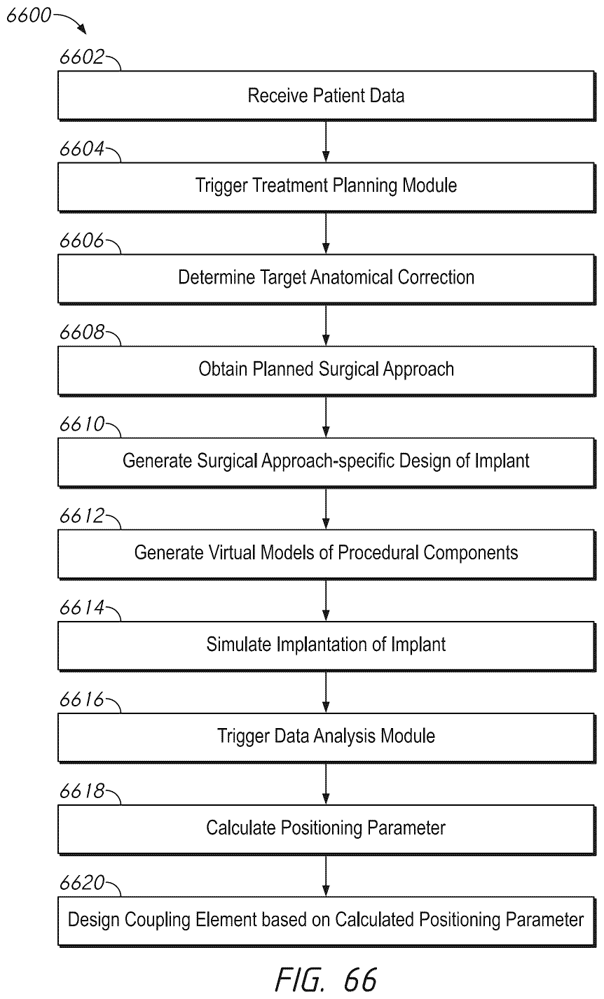

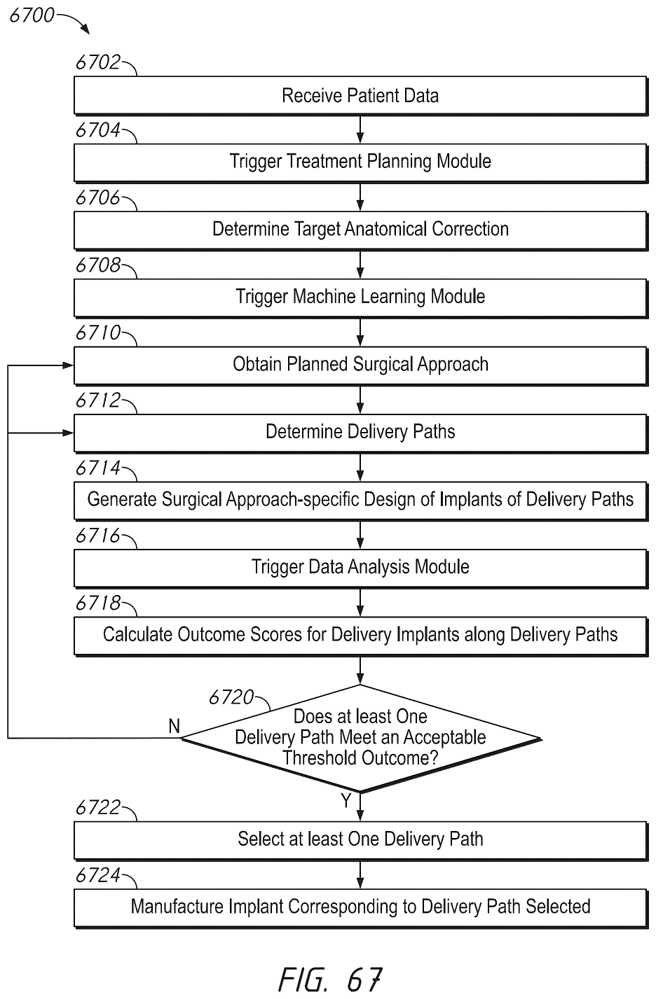

At step 6514, the computing system generates a surgical approach-specific design of an implant. For example, the implant can be one of the implants 2600, 3200, 3800, 4500, 4900, and 5500 described with reference to FIGS. 25-64, or any other implants (e.g., patient-specific or custom implants) or implant components described herein. As described above, the computing system can determine a target location to position the implant based on the target anatomical correction. In some embodiments, the computing system can generate the surgical approach-specific design of the implant for delivery to the target location. In some embodiments, the implant is configured to detachably couple to an insertion instrument such that the implant is angled with respect to the longitudinal axis of the insertion instrument. For example, when the computing system generates a surgical approach-specific design of the implant, it can additionally generate one or more insertion instruments, coupling elements (i.e., elements that couple the implant to the insertion instrument), and/or additional procedural components associated with an angular orientation to achieve the target anatomical correction using the planned surgical approach. The configuration of the implant and the additional procedural components can enable the implant to achieve the target anatomical correction while the insertion instrument is aligned along a delivery path for the planned surgical approach. In some embodiments, the main body of the implant is designed independently of the connection feature (i.e., the coupling element). For example, the computing system can design the main body and/or features of the implant to achieve the desired anatomical correction and the connection feature to detachably couple to the insertion instrument such that the implant can be delivered along the planned delivery path associated with the surgical approach, as described in more detail with reference to FIGS. 66 and 67.

In some embodiments, the computing system and/or the treatment planning module include an implant footprint database. One or more implant footprints can be used to generate the surgical approach-specific design of the implant. The implant footprint database can include one or more implant footprints (e.g., shape, dimensions, etc.) configured to interface with surrounding tissue or bone at the target anatomical correction. For example, the implant footprint database can include implant parameters such as dimensional design constraints specific to the planned surgical approach of the implant. Additionally, or alternatively, the implant footprint database can be used to design the surface area and shape of the surgical approach-specific design of the implant. By selecting the appropriate implant footprint, the computing system can ensure stability, proper load distribution, and/or other biomechanical requirements of the surgical approach-specific design of the implant.

FIG. 66 is a flow diagram illustrating an example process 6600 for designing a surgical approach-specific implant in accordance with at least some embodiments of the technology. The process 6600 can be performed by a computing system, for example, the computing system 2400 of FIG. 24. As described above, one or more of the steps described herein can be initiated and/or executed by one or more of a user, a computing device (e.g., the computing device 2402 of FIG. 24), or any other computing device in a network of the computing system (e.g., the communication network 2404 of FIG. 24). The process 6600 can be performed in combination with one or more steps of the process 6500 described in connection with FIG. 65 and any of the other processes or methods described herein.

Initially at step 6602, a computing system receives patient data, as described in more detail with reference to step 6502 of FIG. 65. At step 6604, the computing system triggers a treatment planning module, as described in more detail with reference to step 6508 of FIG. 65. At step 6606, the computing system determines a target anatomical correction, for example, based on the patient data received, as described in more detail with reference to step 6510 of FIG. 65. At step 6608, the computing system obtains a planned surgical approach from, for example, a user or a treatment planning module, as described in more detail with reference to step 6512 of FIG. 65. At step 6610, the computing system generates a surgical approach-specific design of an implant based on the target anatomical correction and the planned surgical approach, as described in more detail with reference to step 6514 of FIG. 65.

At step 6612, the computing system generates one or more virtual models of one or more procedural components corresponding to the surgical approach-specific design of the implant. For example, the computing system can generate a virtual model of the target anatomical correction, the implant, an implant insertion instrument, and/or the like. Additionally or alternatively, a plurality of virtual models corresponding to various configurations for the implant, the insertion instrument, and/or the like can be generated to simulate the implantation of multiple surgical approach-specific designs of the implants using different procedural tools and/or delivery paths, as described in more detail below with reference to FIG. 67. In some embodiments, the computing system can generate pre-operative and post-operative models of the target anatomy to visualize the target anatomical correction using patient data and/or procedure data. The virtual models can include an entirety of the patient, for example, a three-dimensional twin of the patient, and/or the virtual models can be limited to just the target anatomy for the anatomical correction (e.g., the patient's spine, etc.).

At step 6614, the computing system simulates implantation of the implant using the one or more virtual models of procedural components generated in step 6612. For example, one or more virtual models of the implant, the insertion instrument, and/or the target anatomical correction can be used to simulate the implantation of the implant at the target location using the obtained surgical approach. In some embodiments, the computing system simulates implantation of different surgical approach-specific designs of the implant using one or more virtual implants, virtual insertion instruments, and/or virtual models of the target anatomical correction. In some embodiments, the implant is detachably coupled to the insertion instrument at an angle relative to the longitudinal axis of the insertion instrument, and one or more angular orientations of the implant relative to the longitudinal axis of the insertion instrument can be modeled virtually to simulate the implantation of the implant using the insertion instrument at various angular orientations.

In some embodiments, one or more patient images, virtual models, delivery paths (e.g., overlaid on patient images, in virtual models, etc.), simulations, and associated metrics/parameters, including pre-operative and post-operative metrics/parameters, can be displayed for user review and/or user feedback on the user device. The computing system can modify one or more elements of the virtual models based on the user's feedback, and an optional additional simulation of implantation can be performed.

At step 6616, the computing system triggers a data analysis module (e.g., the data analysis module 2416 of FIG. 24), as described in more detail with reference to step 6504 of FIG. 65. At step 6618, the computing system calculates a positioning parameter using, for example, the data analysis module. In some embodiments, the positioning parameter is a parameter indicative of an implant-insertion instrument relationship. For example, the data analysis module can determine one or more positioning parameters in the simulations by calculating one or more parameters associated with the virtual implant and/or the virtual insertion instrument. Additionally or alternatively, the data analysis module can calculate the position and/or angular orientation of the virtual model of the insertion instrument relative to the virtual model of the target anatomical correction. In some embodiments, the positioning parameter is an angle calculated between the longitudinal axis of the virtual model of the insertion instrument and an imaginary reference plane (e.g., a transverse plane) of the virtual model of the implant.

In some embodiments, the computing system can determine the implant-insertion instrument relationship after identifying the target anatomical correction for the subject. For example, the implant-insertion instrument relationship can be based on the target location and a virtual anatomical model of the subject after generating an initial surgical approach-specific design of the implant and simulating the implantation. Additionally or alternatively, the implant-insertion instrument relationship can be based on the target location and the virtual anatomical model of the subject, and the implant-insertion instrument relationship can guide the generation of the surgical approach-specific design of the implant. Thus, the computing system can generate the surgical approach-specific design of the implant before and/or after simulating implantation of the implant at the target location.

At step 6620, the computing system designs a coupling element based on the calculated positioning parameter. As described herein, the coupling element can be configured to detachably couple the insertion instrument to the implant such that, for example, the longitudinal axis of the insertion instrument is angled away from the transverse axis of the implant. The configuration of the coupling element can prevent the insertion instrument from contacting the vertebrae of the subject when positioning the implant at the target position. In some embodiments, the coupling element of the implant is an internally threaded hole configured to receive a threaded distal end of the insertion instrument, as described in more detail with reference to FIGS. 30 and 31.

One or both of the position of the coupling element and the angular orientation of the coupling element can be designed based on a planned delivery path corresponding to the planned surgical approach. In some embodiments, one or more virtual models of the coupling element are generated, and one or more additional simulations for implanting the implant are conducted to verify that the angular orientation provided by the coupling element achieves implantability and/or target anatomical correction using the planned delivery path and planned surgical approach. Furthermore, the computing system can analyze the simulations performed using, for example, the data analysis module to identify one or more of the implants, insertion instruments, surgical approaches, delivery paths, coupling elements, etc. associated with the highest threshold outcome score. Additionally or alternatively, the computing system can generate a surgical plan including the procedural elements corresponding to the highest threshold outcome score, as described in more detail with reference to FIG. 67.

FIG. 67 is a flow diagram illustrating an example process 6700 for designing a surgical approach-specific implant in accordance with at least some embodiments of the technology. The process 6700 can be performed by a computing system, for example, the computing system 2400 of FIG. 24. As described above, one or more of the steps described herein can be initiated and/or executed by one or more of a user, a computing device (e.g., the computing device 2402 of FIG. 24), or any other computing device in a network of the computing system (e.g., the communication network 2404 of FIG. 24). The process 6700 can be performed in combination with one or more steps of the processes 6500, 6600 described in FIGS. 65 and 66, respectively, and any of the other processes or methods described herein.

At step 6702, a computing system receives patient data, as described in steps 6502 and 6602 of FIGS. 65 and 66, respectively. At step 6704, the computing system triggers a treatment planning module, as described in steps 6508 and 6604 of FIGS. 65 and 66, respectively. At step 6706, the computing system determines a target anatomical correction based on the received patient data, as described in steps 6510 and 6606 of FIGS. 65 and 66, respectively.

At step 6708, the computing system triggers a machine learning module. The machine learning module can include one or more machine learning models trained on historical patient and/or procedure data. In some embodiments, the machine learning module processes and analyzes the received patient data (e.g., the patient data received at step 6702) to obtain a planned surgical approach and/or the surgical approach-specific design of the implant. Additionally or alternatively, the machine learning module can process and analyze imputing patient data (e.g., imputing image data) to fill in incomplete or missing data from the patient dataset. For example, the machine learning module enables the computing system to process patient images that are corrupted, obscured, and/or missing image data. In some embodiments, the machine learning models can be trained to predict and fill in missing data from the images such that the images received can be used to plan the surgical approach and/or the surgical approach-specific design of the implant. It is worth noting that although in the process 6700 the target anatomical correction is determined prior to triggering the machine learning module, the machine learning module can further process the patient data received to determine the target anatomical correction based on historical patient data. In some embodiments, the machine learning module is a part of the treatment planning module and one or more of the target anatomical correction, surgical approach, delivery paths, implants, and/or the like are determined and/or generated based on historical patient and/or procedure data.

At step 6710, the computing system obtains a planned surgical approach, as described in more detail with reference to steps 6512 and 6608 of FIGS. 65 and 66, respectively. In some embodiments, the machine learning module can predict one or more surgical approaches for delivering the implant. For example, the machine learning module can rank the surgical approaches based on associated outcome scores. The computing system can then notify the user of the ranked surgical approaches. In some embodiments, the user selects a surgical approach by simulating one or more surgical procedures on a virtual model of the patient. Additionally or alternatively, the machine learning module can automatically select the surgical approach by comparing the received patient data to historical patient data and choosing the approach associated with the highest outcome score.

At step 6712, the computing system determines one or more delivery paths, each to a respective level along the patient's target anatomy (e.g., the spine). In some embodiments, the machine learning module is trained on historical patient data and/or procedure data to generate multiple delivery paths corresponding to the planned surgical approach. Additionally or alternatively, the machine learning module can generate the delivery paths based on historical patient data and/or procedure data associated with the physician who will perform the implantation.

At step 6714, the computing system generates a surgical approach-specific design of implants based on the delivery paths for the target anatomical correction and the planned surgical approach, as described in more detail with reference to steps 6514 and 6610 of FIGS. 65 and 66, respectively. For example, the machine learning module can be trained based on implant footprint data to design a plurality of implants and/or procedural components (e.g., insertion instruments, coupling elements, etc.), each configured for delivery along a respective one of the delivery paths.

At step 6716, the computing system triggers a data analysis module (e.g., the data analysis module 2416 of FIG. 24), as described in more detail with reference to steps 6504 and 6616 of FIGS. 65 and 66, respectively. For example, the data analysis module can calculate one or more outcome scores associated with the delivery paths and corresponding implants. The outcome scores can be representative of the success of the surgical procedure (e.g., the likelihood of successfully delivering the implant to the target location, likelihood of achieving the target anatomical correction, etc.) using the delivery path and/or planned surgical approach.

At step 6718, the computing system calculates one or more outcome scores for delivering the implants along the one or more delivery paths generated. At step 6720, the computing system determines whether at least one of the delivery paths meets an acceptable threshold outcome. For example, the computing system can simulate one or more surgical procedures to determine whether the planned surgical approach, delivery path, and/or implant achieves the target anatomical correction and/or achieves an acceptable threshold outcome score.

If none of the delivery paths meets an acceptable threshold outcome, the computing system can return to step 6712 to generate one or more additional delivery paths that can be used to generate one or more surgical approach-specific designs of implants. The computing system can calculate one or more additional outcome scores associated with delivering the additional implants along the additional delivery paths. Additionally or alternatively, the computing system can return to step 6710 to obtain a different planned surgical approach to generate one or more additional delivery paths using a different surgical approach. This allows the computing system to calculate which surgical approach, delivery path, implant, and/or the like have the highest outcome scores, thereby improving patient outcomes.

If at least one delivery path meets an acceptable threshold outcome, then at step 6722, the computing system selects at least one of the delivery paths that meet an acceptable threshold outcome. For example, the computing system can automatically select the delivery path with the highest acceptable threshold outcome score. Additionally or alternatively, the computing system can notify a user to select at least one of the delivery paths that meet an acceptable threshold outcome via the user device.

In some embodiments, the computing system can generate a surgical plan including the procedural elements associated with the delivery path selected. The surgical plan can be generated, at least in part, by identifying one or more steps performed virtually in the simulation of implantation. Each of the steps can be linked to one or more procedural elements (e.g., implants, insertion instruments, coupling elements, etc.). Additionally or alternatively, the surgical plan can be identified using historical surgical plans associated with the surgical procedure to be performed. For example, the computing system can reference past procedures that are similar to the surgical procedure to be performed. In some embodiments, the computing system references past surgical procedures performed by the physician to generate the surgical plan according to the physician's historical outcomes and/or preferences.

At step 6724, the implant associated with the selected delivery path is manufactured. For example, the computing system can trigger a manufacturing system (e.g., the manufacturing system 2424 of FIG. 24) to produce the surgical approach-specific design of the implant. Additionally or alternatively, the computing system can instruct the manufacturing system to create additional procedural components associated with the selected delivery path, such as insertion instruments, coupling elements, and/or the like.

Several aspects of the present technology are set forth in the following examples:

1. A spinal fusion system comprising:
an intervertebral implant including
   a fusion body configured to contact endplates of adjacent vertebrae; and
   an instrument coupler including an instrument-receiving feature configured to be detachably couplable to an inserter instrument, wherein the instrument coupler is configured to be locked to the fusion body when the inserter instrument extends through the instrument-receiving feature and contacts the fusion body, wherein when the inserter instrument is spaced apart from the fusion body and coupled to the instrument coupler, the instrument coupler is movable from a delivery position to an implant position to rotate the fusion body relative to the inserter instrument.

2. The spinal fusion system of example 1, further comprising an inserter instrument including a threaded distal portion positionable in the instrument-receiving feature to be detachably coupled to the intervertebral implant, wherein the intervertebral implant is configured to rotate across a midline of a patient's disc space when the inserter instrument is positioned at a transforaminal path extending from the disc space.

3. The spinal fusion system of any of examples 1-2, further comprising an inserter instrument configured to lock the intervertebral implant to rigidly holding a proximal end of the intervertebral implant and to unlock the intervertebral implant in-vivo to allow a distal end of the intervertebral implant to rotate along a patient's disc space.

4. The spinal fusion system of any of examples 1-3, wherein
the instrument-receiving feature includes a threaded throughhole, and
the fusion body includes
   a first locking face that extends across an opening of the threaded throughhole when the instrument coupler in at the delivery position, and
   a second locking face that extends across the opening of the threaded throughhole when the instrument coupler in at the implant position.

5. The spinal fusion system of any of examples 1-4, wherein the fusion body is configured to arc through about at least 30 degrees relative to the instrument coupler when the intervertebral implant is in an unlocked state.

6. The spinal fusion system of any of examples 1-5, wherein the instrument coupler is slidable along one or more tracks of the fusion body to rotate the fusion body along a transverse plane of the intervertebral implant.

7. The spinal fusion system of any of examples 1-6, wherein the fusion body includes a pair of arcuate tracks along which the instrument coupler slides from the delivery position to the implant position.

8. The spinal fusion system of any of examples 1-8, wherein the fusion body is a C-shaped interbody fusion cage.

9. The spinal fusion system of any of examples 1-9, wherein the fusion body has a longitudinal axis passing through the instrument-receiving feature when the instrument coupler is in the delivery position, and wherein the longitudinal axis is spaced apart from the instrument coupler when the instrument coupler is in the implant position.

11. A method of implanting an intervertebral implant, the method comprising:
delivering an intervertebral implant rigidly coupled to an inserter instrument into an intervertebral disc space of a patient;
unlocking the intervertebral implant using the inserter instrument while the intervertebral implant is at least partially positioned in the intervertebral disc space;
rotating the unlocked intervertebral implant laterally relative to the inserter instrument to reposition the intervertebral implant in the intervertebral disc space; and
separating the inserter instrument from the unlocked intervertebral implant at the intervertebral disc space.

12. The method of example 11, wherein the intervertebral implant includes a fusion body and an instrument coupler that is movable from a delivery position to an implant position when the intervertebral implant is unlocked.

13. The method of any of examples 11-12, further comprising selectively locking and unlocking the intervertebral implant using the inserter instrument while the inserter instrument is connected to intervertebral implant.

14. The method of any of examples 11-13, wherein the intervertebral implant includes a fusion body and an instrument coupler, wherein the method further comprises:
positioning the inserter instrument through an instrument-receiving feature of the instrument coupler such that the inserter instrument contacts the fusion body to lock the intervertebral implant in a delivery configuration; and
rotating the inserter instrument relative to the instrument coupler to move the inserter instrument away from the fusion body to unlock the intervertebral implant.

15. The method of any of examples 11-14, further comprising moving the instrument coupler of the unlocked intervertebral implant from a delivery position to an implant position to rotate the fusion body relative to the delivery instrument.

16. The method of any of examples 11-15, wherein the intervertebral implant extends across a midsagittal plane of the patient and the inserter instrument is spaced apart from the midsagittal plane when the inserter instrument is separated from the intervertebral implant.

17. The method of any of examples 11-16, further comprising rotating the intervertebral implant across a midline of the patient's disc space while the inserter instrument is positioned at a transforaminal path to the disc space.

18. The method of any of examples 11-17, further comprising using the inserter instrument to unlock the intervertebral implant such that a distal end of the intervertebral implant is rotatable in an anterior direction along a patient's disc space.

19. A computer-readable storage medium storing a set of instructions that, when executed by one or more processors, cause the one or more processors to perform a process comprising:
delivering an intervertebral implant rigidly coupled to an inserter instrument into an intervertebral disc space of a patient;
unlocking the intervertebral implant using the inserter instrument while the intervertebral implant is at least partially positioned in the intervertebral disc space;
rotating the unlocked intervertebral implant laterally relative to the inserter instrument to reposition the intervertebral implant in the intervertebral disc space; and
separating the inserter instrument from the unlocked intervertebral implant at the intervertebral disc space.

20. The computer-readable storage medium of example 19, wherein the intervertebral implant includes a fusion body and an instrument coupler that is movable from a delivery position to an implant position when the intervertebral implant is unlocked

21. The computer-readable storage medium of any of examples 19-20, the process further comprising selectively locking and unlocking the intervertebral implant using the inserter instrument while the inserter instrument is connected to intervertebral implant.

22. The computer-readable storage medium of any of examples 19-21, the process further comprising
positioning the inserter instrument through an instrument-receiving feature of the instrument coupler such that the inserter instrument contacts the fusion body to lock the intervertebral implant in a delivery configuration; and
rotating the inserter instrument relative to the instrument coupler to move the inserter instrument away from the fusion body to unlock the intervertebral implant.

23. The computer-readable storage medium of example 22, further comprising moving the instrument coupler of the unlocked intervertebral implant from a delivery position to an implant position to rotate the fusion body relative to the delivery instrument.

24. The computer-readable storage medium of any of examples 19-23, wherein the intervertebral implant extends across a midsagittal plane of the patient and the inserter instrument is spaced apart from the midsagittal plane when the inserter instrument is separated from the intervertebral implant.

25. The computer-readable storage medium of any of examples 19-24, further comprising rotating the intervertebral implant across a midline of the patient's disc space while the inserter instrument is positioned at a transforaminal path to the disc space.

26. The computer-readable storage medium of any of examples 19-25, further comprising using the inserter instrument to unlock the intervertebral implant such that a distal end of the intervertebral implant is rotatable in an anterior direction along a patient's disc space.

27. A computing system for verifying compliance with one or more controls, the computing system comprising:
one or more processors, and
one or more memories storing instructions that, when executed by the one or more processors, cause the computing system to perform a process comprising:
   delivering an intervertebral implant rigidly coupled to an inserter instrument into an intervertebral disc space of a patient;
   unlocking the intervertebral implant using the inserter instrument while the intervertebral implant is at least partially positioned in the intervertebral disc space;
   rotating the unlocked intervertebral implant laterally relative to the inserter instrument to reposition the intervertebral implant in the intervertebral disc space; and
   separating the inserter instrument from the unlocked intervertebral implant at the intervertebral disc space.

28. The computing system of example 27, wherein the intervertebral implant includes a fusion body and an instrument coupler that is movable from a delivery position to an implant position when the intervertebral implant is unlocked

29. The computing system of any of examples 27-28, the process further comprising selectively locking and unlocking the intervertebral implant using the inserter instrument while the inserter instrument is connected to intervertebral implant.

30. The computing system of any of examples 27-29, the process further comprising
positioning the inserter instrument through an instrument-receiving feature of the instrument coupler such that the inserter instrument contacts the fusion body to lock the intervertebral implant in a delivery configuration; and
rotating the inserter instrument relative to the instrument coupler to move the inserter instrument away from the fusion body to unlock the intervertebral implant.

31. The computing system of example 30, further comprising moving the instrument coupler of the unlocked intervertebral implant from a delivery position to an implant position to rotate the fusion body relative to the delivery instrument.

32. The computing system of any of examples 27-31, wherein the intervertebral implant extends across a midsagittal plane of the patient and the inserter instrument is spaced apart from the midsagittal plane when the inserter instrument is separated from the intervertebral implant.

33. The computing system of any of examples 27-32, further comprising rotating the intervertebral implant across a midline of the patient's disc space while the inserter instrument is positioned at a transforaminal path to the disc space.

34. The computing system of any of examples 27-33, further comprising using the inserter instrument to unlock the intervertebral implant such that a distal end of the intervertebral implant is rotatable in an anterior direction along a patient's disc space.

35. A method of designing an implant, the method comprising:
determining a planned in-vivo rotation of an implant based on a target anatomical correction and a planned delivery path; and
designing the implant to rotate relative to an instrument the planned in-vivo rotation such that the implant is moved to a target position for the target anatomical correction while the instrument is positioned along the planned delivery path.

36. The method of example 35, further comprising receiving the planned delivery path from a user.

37. The method of any of examples 35-36, wherein the planned in-vivo rotation occurs prior to detaching the implant from the instrument.

38. The method of any of examples 35-37, wherein the implant has an internal joint configured to allow articulation of the implant while the implant is positioned in an intervertebral space.

39. The method of any of examples 35-38, wherein the implant has an atraumatic end configured to slide along tissue to cause the in-vivo rotation.

40. The method of any of examples 35-39 wherein the implant is designed to automatically move toward the target position based on the instrument being advanced distally.

41. The method of any of examples 35-40, wherein the planned delivery path is a transforaminal delivery path.

42. The method of any of examples 35-41, wherein the implant is designed based on a simulation comprising:
delivering the implant rigidly coupled to the instrument into an intervertebral disc space;
unlocking the implant using the instrument while the implant is at least partially positioned in the intervertebral disc space;
rotating the unlocked implant laterally relative to the instrument to reposition the implant in the intervertebral disc space; and
separating the instrument from the unlocked implant at the intervertebral disc space.

43. A computer-readable storage medium storing a set of instructions that, when executed by one or more processors, cause the one or more processors to perform a process comprising:
determining a planned in-vivo rotation of an implant based on a target anatomical correction and a planned delivery path; and
designing the implant to rotate relative to an instrument the planned in-vivo rotation such that the implant is moved to a target position for the target anatomical correction while the instrument is positioned along the planned delivery path.

44. The computer-readable storage medium of example 43, further comprising receiving the planned delivery path from a user.

45. The computer-readable storage medium of any of examples 43-44, wherein the planned in-vivo rotation occurs prior to detaching the implant from the instrument.

46. The computer-readable storage medium of any of examples 43-45, wherein the implant has an internal joint configured to allow articulation of the implant while the implant is positioned in an intervertebral space.

47. The computer-readable storage medium of any of examples 43-46, wherein the implant has an atraumatic end configured to slide along tissue to cause the in-vivo rotation.

48. The computer-readable storage medium of any of examples 43-47, wherein the implant is designed to automatically move toward the target position based on the instrument being advanced distally.

49. The computer-readable storage medium of any of examples 43-49, wherein the planned delivery path is a transforaminal delivery path.

50. The computer-readable storage medium of any of examples 43-49, wherein the implant is designed based on a simulation comprising:
delivering the implant rigidly coupled to the instrument into an intervertebral disc space;
unlocking the implant using the instrument while the implant is at least partially positioned in the intervertebral disc space;
rotating the unlocked implant laterally relative to the instrument to reposition the implant in the intervertebral disc space; and
separating the instrument from the unlocked implant at the intervertebral disc space.

51. A computing system for verifying compliance with one or more controls, the computing system comprising:
one or more processors, and
one or more memories storing instructions that, when executed by the one or more processors, cause the computing system to perform a process comprising:
   determining a planned in-vivo rotation of an implant based on a target anatomical correction and a planned delivery path; and
   designing the implant to rotate relative to an instrument the planned in-vivo rotation such that the implant is moved to a target position for the target anatomical correction while the instrument is positioned along the planned delivery path.

52. The computing system of example 51, further comprising receiving the planned delivery path from a user.

53. The computing system of any of examples 51-52, wherein the planned in-vivo rotation occurs prior to detaching the implant from the instrument.

54. The computing system of any of examples 51-53, wherein the implant has an internal joint configured to allow articulation of the implant while the implant is positioned in an intervertebral space.

55. The computing system of any of examples 51-54, wherein the implant has an atraumatic end configured to slide along tissue to cause the in-vivo rotation.

56. The computing system of any of examples 51-55, wherein the implant is designed to automatically move toward the target position based on the instrument being advanced distally.

57. The computing system of any of examples 51-56, wherein the planned delivery path is a transforaminal delivery path.

58. The computing system of any of examples 51-57, wherein the implant is designed based on a simulation comprising:
delivering the implant rigidly coupled to the instrument into an intervertebral disc space;
unlocking the implant using the instrument while the implant is at least partially positioned in the intervertebral disc space;
rotating the unlocked implant laterally relative to the instrument to reposition the implant in the intervertebral disc space; and
separating the instrument from the unlocked implant at the intervertebral disc space.

59. A method comprising:
determining a target anatomical correction for a subject;
obtaining a planned surgical approach; and
generating a surgical approach-specific design of an implant based on the target anatomical correction and the planned surgical approach, wherein the implant is configured to detachably couple to an insertion instrument such that the implant is angled away from a longitudinal axis of the insertion instrument for positioning the implant at a target location while the insertion instrument is positioned along a delivery path for the planned surgical approach.

60. The method of example 59, further comprising:
selecting an implant footprint from an implant footprint database; and
using the implant footprint to generate the surgical approach-specific design of the implant.

61. The method of example 60, wherein the implant footprint includes one or more dimensional design constraints.

62. The method of example 60, wherein the implant footprint includes a shape for the surgical approach-specific design.

63. The method of any of examples 59-62, further comprising:
simulating implantation of the implant using a virtual implant and a virtual anatomical model of the subject; and
designing a coupling element of the implant configured to detachably couple to the insertion instrument based on a position of a virtual model of the insertion instrument relative to the virtual anatomical model of the subject.

64. The method of example 63, wherein designing the coupling element includes determining
a position for the coupling element; and
an orientation for the coupling element based on the delivery path.

65. The method of any of examples 59-64, further comprising:
determining target location for the implant based on the target anatomical correction; and
designing the implant for delivery to the target location.

66. The method of any of examples 59-65, further comprising designing an angled coupling element of the implant configured to detachably receive the insertion instrument such that the longitudinal axis of the insertion instrument is angled away from a transverse plane of the implant.

67. The method of any of examples 59-66, further comprising determining an angle between an axis of an angled coupling element configured to couple to the insertion instrument and an imaginary reference plane of the implant based on delivery to the target location.

68. The method of any of examples 59-67, wherein the implant includes an angled coupling element including an internally threaded hole configured to receive a threaded distal end of the insertion instrument.

69. The method of any of examples 59-68, wherein the planned surgical approach is obtained from a machine learning module trained to generate delivery paths based on historical patient data.

70. The method of any of examples 59-69, further comprising:
determining a plurality of delivery paths each to a respective level along the subject's spine; and
designing a plurality of implants each configured for delivery along a respective one of the delivery paths.

71. The method of any of examples 59-70, wherein the planned surgical approach is obtained from a user device of a physician.

72. The method of any of examples 59-71, further comprising determining an implant-insertion instrument relationship based on the target location and at least one of the virtual anatomical model of the subject or a target anatomical correction for the subject, wherein generation of the surgical approach-specific design of the implant is based on the implant-insertion instrument relationship.

73. The method of example 72, wherein the implant-insertion instrument relationship is determined after determining the target anatomical correction for the subject.

74. The method of example 72, wherein the implant-insertion instrument relationship includes an angle between a reference plane of the implant and the longitudinal axis of the insertion instrument.

75. The method of any of examples 59-74, wherein generating the surgical approach-specific design of the implant includes designing a main body of the implant to achieve the target anatomical correction; and designing a connection feature of the implant such that the insertion instrument is detachably couplable to the connection feature for delivering the implant generally along the delivery path.

76. The method of example 75, wherein the main body is designed independent of the connection feature.

77. The method of example 75, further comprising design a coupling feature of the implant configured to couple to the insertion instrument so to prevent the insertion instrument from contacting vertebrae of the subject when positioning the implant at the target location.

78. The method of any of examples 59-77, further comprising:
determining whether the delivery path meets an acceptable outcome threshold; and
manufacturing the implant after determining that the delivery path meets the acceptable outcome threshold.

79. The method of any of examples 59-78, further comprising simulating one or more surgical procedures for achieving the target anatomical correction to determine whether the planned surgical approach meets an acceptable outcome threshold.

80. The method of any of examples 59-79, further comprising simulating one or more surgical procedures on the subject to determine the planned surgical approach.

81. The method of any of examples 59-80, further comprising:
training a machine learning module using historical patient data;
inputting imputing image data of the subject into the trained machine learning module; and
obtaining the planned surgical approach and design of the implant from the trained machine learning module.

82. The method of any of examples 59-81, further comprising determining the delivery path to the target location based on historical patient data associated with a physician selected to implant the implant.

83. A computer-readable storage medium storing a set of instructions that, when executed by one or more processors, cause the one or more processors to perform any one of examples 59-82.

84. A computing system for verifying compliance with one or more controls, the computing system comprising:
one or more processors, and
one or more memories storing instructions that, when executed by the one or more processors, cause the computing system to perform any one of examples 59-82.

Various methods and techniques described above provide a number of ways to carry out the invention. Of course, it is to be understood that not necessarily all objectives or advantages described may be achieved in accordance with any particular embodiment described herein and may depend on the use of the mounting systems. Thus, for example, those skilled in the art will recognize that the methods may be performed in a manner that achieves or optimizes one advantage or group of advantages as taught herein without necessarily achieving other objectives or advantages as may be taught or suggested herein. Furthermore, the skilled artisan will recognize the interchangeability of various features from different embodiments disclosed herein. Similarly, the various features and acts discussed above, as well as other known equivalents for each such feature or act, can be mixed and matched by one of ordinary skill in this art to perform methods in accordance with principles described herein.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2018, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;” and
U.S. application Ser. No. 16/699,447, filed Nov. 29, 2019, titled “SYSTEMS AND METHODS FOR ORTHOPEDIC IMPLANTS;”
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. application Ser. No. 17/342,439, filed Jun. 8, 2021, titled “PATIENT-SPECIFIC MEDICAL PROCEDURES AND DEVICES, AND ASSOCIATED SYSTEMS AND METHODS;”
U.S. application Ser. No. 17/463,054, filed Aug. 31, 2021, titled “BLOCKCHAIN MANAGED MEDICAL IMPLANTS;”
U.S. application Ser. No. 17/518,524, filed Nov. 3, 2021, titled “PATIENT-SPECIFIC ARTHROPLASTY DEVICES AND ASSOCIATED SYSTEMS AND METHODS;”
U.S. application Ser. No. 17/531,417, filed Nov. 19, 2021, titled “PATIENT-SPECIFIC JIG FOR PERSONALIZED SURGERY;”
U.S. application Ser. No. 17/678,874, filed Feb. 23, 2022, titled “NON-FUNGIBLE TOKEN SYSTEMS AND METHODS FOR STORING AND ACCESSING HEALTHCARE DATA;”
U.S. application Ser. No. 17/835,777, filed Jun. 8, 2022, titled “PATIENT-SPECIFIC EXPANDABLE INTERVERTEBRAL IMPLANTS;”
U.S. application Ser. No. 17/842,242, filed Jun. 16, 2022, titled “PATIENT-SPECIFIC ANTERIOR PLATE IMPLANTS;”
U.S. application Ser. No. 17/851,487, filed Jun. 28, 2022, titled “PATIENT-SPECIFIC ADJUSTMENT OF SPINAL IMPLANTS, AND ASSOCIATED SYSTEMS AND METHODS;”
U.S. application Ser. No. 17/856,625, filed Jul. 1, 2022, titled “SPINAL IMPLANTS FOR MESH NETWORKS;”
U.S. application Ser. No. 17/867,621, filed Jul. 18, 2022, titled “PATIENT-SPECIFIC SACROILIAC IMPLANT, AND ASSOCIATED SYSTEMS AND METHODS;”
U.S. application Ser. No. 17/868,729, filed Jul. 19, 2022, titled “SYSTEMS FOR PREDICTING INTRAOPERATIVE PATIENT MOBILITY AND IDENTIFYING MOBILITY-RELATED SURGICAL STEPS;”
U.S. application Ser. No. 17/951,085, filed Sep. 22, 2022, titled “SYSTEMS FOR MANUFACTURING AND PRE-OPERATIVE INSPECTING OF PATIENT-SPECIFIC IMPLANTS;”
U.S. application Ser. No. 17/978,673, filed Nov. 1, 2022, titled “SPINAL IMPLANTS AND SURGICAL PROCEDURES WITH REDUCED SUBSIDENCE, AND ASSOCIATED SYSTEMS AND METHODS;”
U.S. application Ser. No. 17/978,746, filed Nov. 1, 2022, titled “PATIENT-SPECIFIC SPINAL INSTRUMENTS FOR IMPLANTING IMPLANTS AND DECOMPRESSION PROCEDURES;”
U.S. application Ser. No. 18/102,444, filed Jan. 27, 2023, titled “TECHNIQUES TO MAP THREE-DIMENSIONAL HUMAN ANATOMY DATA TO TWO-DIMENSIONAL HUMAN ANATOMY DATA;”
U.S. application Ser. No. 18/113,573, filed Feb. 24, 2023, titled “PATIENT-SPECIFIC IMPLANT DESIGN AND MANUFACTURING SYSTEM WITH A DIGITAL FILING CABINET;”
U.S. application Ser. No. 18/120,979, filed Mar. 13, 2023, titled “MULTI-STAGE PATIENT-SPECIFIC SURGICAL PLANS AND SYSTEMS AND METHODS FOR CREATING AND IMPLEMENTING THE SAME;”
U.S. application Ser. No. 18/455,881, filed Aug. 25, 2023, titled “SYSTEMS AND METHODS FOR GENERATING MULTIPLE PATIENT-SPECIFIC SURGICAL PLANS AND MANUFACTURING PATIENT-SPECIFIC IMPLANTS;”
U.S. application Ser. No. 18/384,762, filed Oct. 28, 2023, titled “SYSTEMS AND METHODS FOR SELECTING, REVIEWING, MODIFYING, AND/OR APPROVING SURGICAL PLANS;”
U.S. application Ser. No. 18/537,600, filed Dec. 12, 2023, titled “PATIENT-SPECIFIC IMPLANT DESIGN AND MANUFACTURING SYSTEM WITH A REGULATORY AND REIMBURSEMENT MANAGER;”
International Application No. PCT/US24/10202, filed Jan. 3, 2024, titled “PATIENT-SPECIFIC SPINAL FUSION DEVICES AND ASSOCIATED SYSTEMS AND METHODS;”
U.S. application Ser. No. 18/408,409, filed Jan. 9, 2024, titled “SYSTEM FOR EDGE CASE PATHOLOGY IDENTIFICATION AND IMPLANT MANUFACTURING;”
U.S. application Ser. No. 18/408,452, filed Jan. 9, 2024, titled “SYSTEM FOR MODELING PATIENT SPINAL CHANGES;”
U.S. application Ser. No. 18/415,577, filed Jan. 17, 2024, titled “PATIENT-SPECIFIC IMPLANT DESIGN AND MANUFACTURING SYSTEM WITH A SURGICAL IMPLANT POSITIONING MANAGER;”
U.S. Application No. 63/539,797, filed Sep. 21, 2023, titled “ROTATABLE INTERVERTEBRAL IMPLANTS FOR TRANSFORAMINAL LUMBAR INTERBODY FUSION TECHNIQUES;” and
U.S. Application No. 63/542,264, filed Oct. 3, 2023, titled “PATIENT-SPECIFIC SURGICAL POSITIONING GUIDES AND METHODS OF MAKING AND USING THE SAME.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, it is not intended that the invention be limited, except as by the appended claims.

1. A spinal fusion system comprising:
an intervertebral implant including
a fusion body configured to contact endplates of adjacent vertebrae; and
an instrument coupler including an instrument-receiving feature configured to be detachably couplable to an inserter instrument, wherein the instrument coupler is configured to be locked to the fusion body when the inserter instrument extends through the instrument-receiving feature and contacts the fusion body, wherein when the inserter instrument is spaced apart from the fusion body and coupled to the instrument coupler, the instrument coupler is movable from a delivery position to an implant position to rotate the fusion body relative to the inserter instrument.

2. The spinal fusion system of claim 1, further comprising an inserter instrument including a threaded distal portion positionable in the instrument-receiving feature to be detachably coupled to the intervertebral implant, wherein the intervertebral implant is configured to rotate across a midline of a patient's disc space when the inserter instrument is positioned at a transforaminal path extending from the disc space.

3. The spinal fusion system of claim 1, further comprising an inserter instrument configured to lock the intervertebral implant to rigidly holding a proximal end of the intervertebral implant and to unlock the intervertebral implant in-vivo to allow a distal end of the intervertebral implant to rotate along a patient's disc space.

4. The spinal fusion system of claim 1, wherein
the instrument-receiving feature includes a threaded throughhole, and
the fusion body includes
a first locking face that extends across an opening of the threaded throughhole when the instrument coupler in at the delivery position, and
a second locking face that extends across the opening of the threaded throughhole when the instrument coupler in at the implant position.

5. The spinal fusion system of claim 1, wherein the fusion body is configured to arc through about at least 30 degrees relative to the instrument coupler when the intervertebral implant is in an unlocked state.

6. The spinal fusion system of claim 1, wherein the instrument coupler is slidable along one or more tracks of the fusion body to rotate the fusion body along a transverse plane of the intervertebral implant.

7. The spinal fusion system of claim 1, wherein the fusion body includes a pair of arcuate tracks along which the instrument coupler slides from the delivery position to the implant position.

8. The spinal fusion system of claim 1, wherein the fusion body is a C-shaped interbody fusion cage.

9. The spinal fusion system of claim 1, wherein the fusion body has a longitudinal axis passing through the instrument-receiving feature when the instrument coupler is in the delivery position, and wherein the longitudinal axis is spaced apart from the instrument coupler when the instrument coupler is in the implant position.

10. The spinal fusion system of claim 1, wherein the instrument coupler is captively held by and moves along a path defined by the fusion body.

11. A method of implanting an intervertebral implant, the method comprising:
delivering an intervertebral implant rigidly coupled to an inserter instrument into an intervertebral disc space of a patient;
unlocking the intervertebral implant using the inserter instrument while the intervertebral implant is at least partially positioned in the intervertebral disc space;
rotating the unlocked intervertebral implant laterally relative to the inserter instrument to reposition the intervertebral implant in the intervertebral disc space; and
separating the inserter instrument from the unlocked intervertebral implant at the intervertebral disc space.

12. The method of claim 11, wherein the intervertebral implant includes a fusion body and an instrument coupler that is movable from a delivery position to an implant position when the intervertebral implant is unlocked.

13. The method of claim 11, further comprising selectively locking and unlocking the intervertebral implant using the inserter instrument while the inserter instrument is connected to intervertebral implant.

14. The method of claim 11, wherein the intervertebral implant includes a fusion body and an instrument coupler, wherein the method further comprises:
positioning the inserter instrument through an instrument-receiving feature of the instrument coupler such that the inserter instrument contacts the fusion body to lock the intervertebral implant in a delivery configuration; and
rotating the inserter instrument relative to the instrument coupler to move the inserter instrument away from the fusion body to unlock the intervertebral implant.

15. The method of claim 14, further comprising moving the instrument coupler of the unlocked intervertebral implant from a delivery position to an implant position to rotate the fusion body relative to the delivery instrument.

16. The method of claim 11, wherein the intervertebral implant extends across a midsagittal plane of the patient and the inserter instrument is spaced apart from the midsagittal plane when the inserter instrument is separated from the intervertebral implant.

17. The method of claim 11, further comprising rotating the intervertebral implant across a midline of the patient's disc space while the inserter instrument is positioned at a transforaminal path to the disc space.

18. The method of claim 11, further comprising using the inserter instrument to unlock the intervertebral implant such that a distal end of the intervertebral implant is rotatable in an anterior direction along a patient's disc space.

19. A computer-readable storage medium storing a set of instructions that, when executed by one or more processors, cause the one or more processors to perform a process comprising:
delivering an intervertebral implant rigidly coupled to an inserter instrument into an intervertebral disc space of a patient;
unlocking the intervertebral implant using the inserter instrument while the intervertebral implant is at least partially positioned in the intervertebral disc space;
rotating the unlocked intervertebral implant laterally relative to the inserter instrument to reposition the intervertebral implant in the intervertebral disc space; and
separating the inserter instrument from the unlocked intervertebral implant at the intervertebral disc space.

20. The computer-readable storage medium of claim 19, wherein the intervertebral implant includes a fusion body and an instrument coupler that is movable from a delivery position to an implant position when the intervertebral implant is unlocked

21. The computer-readable storage medium of claim 19, the process further comprising selectively locking and unlocking the intervertebral implant using the inserter instrument while the inserter instrument is connected to intervertebral implant.

22. The computer-readable storage medium of claim 19, the process further comprising
positioning the inserter instrument through an instrument-receiving feature of an instrument coupler such that the inserter instrument contacts the fusion body to lock the intervertebral implant in a delivery configuration; and
rotating the inserter instrument relative to the instrument coupler to move the inserter instrument away from the fusion body to unlock the intervertebral implant.

23. The computer-readable storage medium of claim 22, further comprising moving the instrument coupler of the unlocked intervertebral implant from a delivery position to an implant position to rotate the fusion body relative to the delivery instrument.

24. The computer-readable storage medium of claim 19, wherein the intervertebral implant extends across a midsagittal plane of the patient and the inserter instrument is spaced apart from the midsagittal plane when the inserter instrument is separated from the intervertebral implant.

25. The computer-readable storage medium of claim 19, further comprising rotating the intervertebral implant across a midline of the patient's disc space while the inserter instrument is positioned at a transforaminal path to the disc space.

26. The computer-readable storage medium of claim 19, further comprising using the inserter instrument to unlock the intervertebral implant such that a distal end of the intervertebral implant is rotatable in an anterior direction along a patient's disc space.

27-82. (canceled)
